(19) United States
(12) Patent Application Publication
(10) Pub. No.: US 2026/0248609 A1
(43) Pub. Date: Aug. 27, 2026

(54) THERMOFORMED POLYMERIC VALVED CONDUITS FOR HEART VALVE APPLICATIONS

(71) Applicants: GEORGIA TECH RESEARCH CORPORATION, Atlanta, GA (US); COLORADO STATE UNIVERSITY RESEARCH FOUNDATION, Fort Collins, CO (US)

(72) Inventors: Srujana JOSHI, Atlanta, GA (US); Hieu BUI, Atlanta, GA (US); Lakshmi Prasad DASI, Atlanta, GA (US); Susan JAMES, Fort Collins, CO (US); Justin GANGWISH, Fort Collins, CO (US)

(21) Appl. No.: 18/875,981

(22) PCT Filed: Jun. 19, 2023
(86) PCT No.: PCT/US2023/025660

§ 371 Date: Dec. 17, 2024

Related U.S. Application Data
(60) Provisional application No. 63/353,443, filed on Jun. 17, 2022.

(51) Int. Cl. A61F 2/24; A61F 2/06; A61L 31/04; A61L 31/12

(52) U.S. Cl. CPC A61F 2/2415; A61F 2/06; A61L 31/048; A61L 31/129; A61F 2210/0071; A61F 2250/0082

(57) ABSTRACT

In accordance with the purposes of the disclosed materials and methods, as embodied and broadly described herein, the disclosed subject matter, in one aspect, relates to a thermoformed polymeric valved conduits for heart valve and methods of using and making thereof. The thermoformed polymeric valved conduits is formed via a thermoforming process that conforms the conduit to a mold; the process forms a conduit that is flexible, resizable, and/or deformable after being formed.

### CROSS-REFERENCE TO RELATED APPLICATIONS

This application claims the benefit of priority to, and the benefit of, U.S. Provisional Application No. 63/353,443 filed on Jun. 17, 2022, the disclosure of which is hereby incorporated by reference herein in its entirety.

### STATEMENT OF GOVERNMENT SUPPORT

This invention was made with government support under Grant No. R01HL135505, awarded by the National Institutes of Health. The government has certain rights in the invention.

### TECHNICAL FIELD

The present disclosure generally relates to methods and systems for using and making thermoformed polymeric conduits or thermoformed polymeric conduits with valves. The thermoformed polymeric conduits or conduits with valves can be configured for applications in which the conduit is expanded as part of its intended use or non-expanded.

### BACKGROUND

Many repair operations for congenital heart defects or other heart defects involve the replacement of valves and/or the implantation of conduits to redirect blood flow. Most surgically implanted valves or conduits are designed to operate in the body for over 10 to 20 years before they wear out, become obstructed, or lose efficiency.

A right ventricle to pulmonary artery (RV-PA) conduit, for example, is a means to supply blood flow to the lungs. It can be used for heart issues such as tetralogy of Fallot, pulmonary atresia, or pulmonary stenosis. RV-PA conduits can also be used as a part of complex surgeries such as the Ross procedure, the Rastelli procedure, or in the Sano modification of the Norwood procedure. Further, they can be used to fix a regurgitant (leaky) or stenotic (narrowed) pulmonary valve or used to replace an absent right ventricular outflow tract.

RV-PA valves have included cryopreserved pulmonary homografts, though these homografts presented issues such as limited availability with appropriate sizing, especially with respect to neonates and infants. Cryopreserved pulmonary homografts also presented problems such as high calcification rates with non-blood-matched homografts, which led to lower durability.

Another option has been xenograft conduits, which vary widely in design, with stented and non-stented options, utilizing bovine and porcine tissues and incorporating conduits made from ePTFE, PET, or animal tissue. These conduits have had problems with endocarditis, calcification, stenosis, and pulmonary insufficiency.

A further option has included bulging sinus ePTFE grafts; however it remains a challenge to produce and rely on the skill of surgeons to hand sew this device. Additionally, the grafts are subject to calcification and neointimal proliferation. A need remains for patient-specific devices that are anti-thrombotic and calcification resistant that also have excellent hemodynamics.

There is a benefit to improving the design and use of implantable conduits and vessels for the right ventricle to pulmonary artery (RV-PA), among other heart conduits and the body's vascular vessels.

### SUMMARY

In accordance with the purposes of the disclosed materials and methods, as embodied and broadly described herein, the disclosed subject matter, in one aspect, relates to a thermoformed polymeric valved conduit for a heart valve and methods of using and making the same. The thermoformed polymeric valved conduit is formed via a thermoforming process that conforms the conduit to a mold. The process can be employed to form either (i) an expandable and resizable conduit that is flexible, deformable, and resizable after being formed or (ii) a non-expandable conduit.

Thus, in one example, a conduit device is provided, comprising a thermoformed conduit comprising a first low melting point thermoplastic material suitable for thermoforming, the thermoformed conduit comprising a first end and a second end and defining a channel therebetween, wherein the conduit device is configured to be surgically implanted in a subject's heart or blood vessel. The conduit device may include at least two layers of thermoplastic material. The thermoplastic material may include additives, e.g., hyaluronan or others described herein, in an interpenetrating network to be anti-thrombotic and/or calcification resistant. The first layer may form leaflets to form a valve, e.g., that functions based on a pressure differential, and the second layer forms a conduit. The layers are then fused or bonded to form a valved conduit.

In a further example, a conduit device is provided, comprising a thermoformed conduit comprising a first low melting point thermoplastic material suitable for thermoforming, the thermoformed conduit comprising a first end and a second end and defining a channel therebetween, the conduit device including sewable rings (also referred to herein as sewing rings) embedded into the first and second ends of the thermoformed conduct to be surgically implanted in a subject's heart or blood vessel.

In further examples, a method of fabricating a conduit device is provided, comprising thermoforming a thermoformed conduit comprising a first low melting point thermoplastic material suitable for thermoforming, the thermoformed conduit comprising a first end and a second end and defining a channel therebetween, wherein the conduit device is configured to be surgically implanted in a subject's heart or blood vessel. The method includes thermoforming the first thermoplastic to form the conduit via a thermal process that conforms the conduit to a first mold; thermoforming the first thermoplastic comprising preheating the first thermoplastic and thermoforming the first thermoplastic on the first mold to form the conduit (e.g., including leaflets); thermoforming a second thermoplastic into the outer layer that conforms the second thermoplastic to a second mold, sewable rings are embedded into at least one of or between first and second thermoplastics. The first and second thermoplastics can be heated to be fused to one another or can be otherwise bonded. The first thermoplastic and the second thermoplastic may be of the same material in one example. In other examples, the first thermoplastic and the second thermoplastic are made of different materials.

Additionally, an expandable conduit device is provided, comprising an expandable thermoformed conduit formed of a first low melting point thermoplastic material suitable of thermoforming, the thermoformed conduit comprising a first end and a second end and defining a channel therebetween, wherein the expandable thermoformed conduit is configured to have (i) a first configuration having a first circumferential profile and (ii) a second configuration having a second circumferential profile, wherein the second circumferential profile has a larger cross-sectional area than the first circumferential profile, wherein the expandable conduit device is configured to be surgically implanted in a subject's heart or blood vessel.

Further, a method is provided comprising providing an expandable thermoformed conduit comprising a low melting point thermoplastic material suitable for thermoforming, the thermoformed conduit comprising a first end and a second end and defining a channel therebetween, wherein the conduit device is configured to be surgically embedded into a subject's heart, the expandable thermoformed conduit configured to have a first configuration having a first circumferential profile at a first position in the channel and a second configuration having a second circumferential profile at the first position, wherein the second circumferential profile has a larger cross-sectional area than the first circumferential profile; implanting the expandable thermoformed conduit while the expandable thermoformed conduit is in the first configuration; and mechanically inducing the expandable thermoformed conduit to the second configuration such that a plastic region of the valved conduit is deformed into an expanded configuration compared to an original expansion; and maintaining the expanded configuration.

In an aspect, a conduit device is disclosed, comprising a thermoformed conduit comprising a first low melting point thermoplastic material suitable for thermoforming, the thermoformed conduit comprising a first end and a second end and defining a channel therebetween, wherein the conduit device is configured to be surgically implanted in a subject's heart or blood vessel.

In some embodiments, the thermoformed conduit comprises a valve, wherein the valve is positioned in the channel or at the second end (e.g., TPV).

In some embodiments, the thermoformed conduit comprises one or more sewable rings, including a first sewable ring, the first sewable ring being coupled to a circumferential portion of the first end (e.g., wherein the first and second sewable rings each comprises a second thermoplastic material, a fabric, etc.).

In some embodiments, the thermoformed conduit comprises an inner layer that is made of the first low melting point thermoplastic material; and an outer layer disposed over the first sewable ring, the second sewable ring, and the conduit such that the first and second sewable rings are fixed between the conduit and the outer layer, wherein the outer layer comprises a third low melting point thermoplastic material.

In some embodiments, the thermoformed conduit comprises a hyaluronan additive (e.g., additive to the first low melting point thermoplastic material).

In some embodiments, at least one of the first low melting point thermoplastic material, the second thermoplastic material, and the third low melting point thermoplastic material comprise polyethylene.

In some embodiments, the polyethylene comprises linear low-density polyethylene (LLDPE).

In some embodiments, at least one of the first low melting point thermoplastic material, the second thermoplastic material, and the third low melting point thermoplastic material comprise polyethylene terephthalate (PET).

In some embodiments, the thermoformed conduit comprises LLDPE and hyaluronan additive arranged in an interpenetrating network.

In some embodiments, at least one of the first low melting point thermoplastic material, the second thermoplastic material, and the third low melting point thermoplastic material is a woven material.

In some embodiments, at least one of the first low melting point thermoplastic materials, the second thermoplastic material, and the third low melting point thermoplastic material is a non-woven material.

In some embodiments, the thermoformed conduit is shaped to form a sinus.

In some embodiments, the thermoformed conduit is calcification resistant or anti-thrombotic.

In some embodiments, the thermoformed conduit is expandable to operate in a first-installed configuration and a second-modified configuration to have an expanded cross-sectional area compared to the first-installed configuration.

In some embodiments, the thermoformed conduit is configured to operate in a non-expandable state.

In another aspect, a method is disclosed of fabricating a conduit device comprising thermoforming a thermoformed conduit comprising a first low melting point thermoplastic material suitable for thermoforming, the thermoformed conduit comprising a first end and a second end and defining a channel therebetween, wherein the conduit device is configured to be surgically implanted in a subject's heart or blood vessel.

In another aspect, a method is disclosed of fabricating a conduit device comprising thermoforming a thermoformed conduit comprising (i) an inner layer comprising a first low melting point thermoplastic material and (ii) an outer layer comprising a second low melting point thermoplastic material each suitable for thermoforming, wherein the thermoformed conduit comprises a first end and a second end and defines a channel therebetween, wherein the conduit device is configured to be surgically implanted in a subject's heart or blood vessel.

In some embodiments, the method includes positioning a first sewable ring and a second sewable ring within structures of the thermoformed conduit, wherein the thermoformed conduit device further comprises an outer layer, wherein the thermoforming embeds the first sewable ring and the second sewable ring within the thermoformed conduit and fuses the inner layer and the outer layer with the first sewable ring and a second sewable ring embedded therebetween.

In some embodiments, the method further includes receiving a topology image or scan of the vessel or heart of the subject; obtaining a topology mapping of the patient's anatomy from the topology image or scan; fabricating one or more molds based on the topology mapping; and thermoforming the thermoformed conduit over the fabricated one or more molds.

In some embodiments, the method further includes determining at least one of a curvature, a diameter, and a length parameter for the one or more molds using the topology image or scan, wherein the at least one curvature, diameter, and length parameter corresponds to a portion of the thermoformed conduit to be thermoformed using the one or more molds.

In another aspect, an expandable conduit device is disclosed comprising an expandable thermoformed conduit formed of a first low melting point thermoplastic material suitable of thermoforming, the thermoformed conduit comprising a first end and a second end and defining a channel therebetween, wherein the expandable thermoformed conduit is configured to have (i) a first configuration having a first circumferential profile and (ii) a second configuration having a second circumferential profile, wherein the second circumferential profile has a larger cross-sectional area than the first circumferential profile, wherein the expandable conduit device is configured to be surgically implanted in a subject's heart or blood vessel.

In some embodiments, the expandable thermoformed conduit is configured to expand to the second circumferential profile via plastic deformation when mechanically induced by an expandable device (e.g., balloon, catheter).

In some embodiments, the expandable thermoformed conduit has a length defined by a longitudinal profile, wherein the first circumferential profile is defined along a portion of the longitudinal profile (ingress, egress, interchannel), wherein the expandable thermoformed conduit comprises a circumferential pleated or folded region at a portion of the first circumferential profile when in the first configuration, the circumferential pleated or circumferential folded region extending along the longitudinal profile or a portion thereof.

In some embodiments, the expandable thermoformed conduit has a length defined by a longitudinal profile, the expandable thermoformed conduit comprising a longitudinal pleated region or longitudinal folded region along the longitudinal profile or a portion thereof.

In some embodiments, the expandable thermoformed conduit has a length defined by a longitudinal profile, wherein the first circumferential profile is defined along a portion of the longitudinal profile (ingress, egress, interchannel), wherein the expandable thermoformed conduit being configured (e.g., designed and thermoformed) comprising: a circumferential pleated region or circumferential folded region at a portion of the first circumferential profile when in the first configuration, the circumferential pleated or circumferential folded region extending along the longitudinal profile or a portion thereof; and a longitudinal pleated region or longitudinal folded region along the longitudinal profile or a portion thereof.

In some embodiments, the device further includes a reinforcing structure disposed within the thermoformed conduit (e.g., between an outer layer and an inner layer).

In some embodiments, the reinforcing structure comprises a stent-shaped structure comprising at least one strut.

In some embodiments, the reinforcing structure comprises a metallic composite or alloy.

In some embodiments, the reinforcing structure is a coil-shaped structure.

In some embodiments, the reinforcing structure comprises a polymer or a thermoplastic material.

In some embodiments, the device includes two or more leaflets configured to operate as a valve by moving collectively between an open state and a closed state, the two or more leaflets each comprising a first end and a second end and defining a curved length therebetween, at least one of the two or more leaflets being sized to (i) form the closed state in connection with other leaflets in the first configuration and (ii) form the closed state in connection with other leaflets in the second configuration.

In another aspect, a method is disclosed (e.g., of operating an expandable conduit device) comprising: providing an expandable thermoformed conduit comprising a low melting point thermoplastic material suitable for thermoforming, the thermoformed conduit comprising a first end and a second end and defining a channel therebetween, wherein the conduit device is configured to be surgically embedded into a subject's heart, the expandable thermoformed conduit configured to have a first configuration having a first circumferential profile at a first position in the channel and a second configuration having a second circumferential profile at the first position, wherein the second circumferential profile has a larger cross-sectional area than the first circumferential profile; implanting the expandable thermoformed conduit while the expandable thermoformed conduit is in the first configuration; and mechanically inducing the expandable thermoformed conduit to the second configuration.

In some embodiments, the operation of mechanically inducing comprises: positioning an expandable member (e.g., balloon, catheter, etc.) in a position within the channel of the expandable thermoformed conduit; and expanding the expandable member to adjust the cross-sectional area of the expandable thermoformed conduit at the first position to the second circumferential profile. The second circumferential profile maintaining the hemodynamic performance as the first circumferential profile.

In some embodiments, the expandable thermoformed conduit is configured to receive a transcatheter pulmonary valve.

In some embodiments, the expandable thermoformed conduit is configured as a right ventricle to pulmonary artery (RV-PA) conduit.

In some embodiments, the first configuration is sized for a child or a young adult.

In some embodiments, the second configuration is sized for a young adult or an adult.

Additional advantages will be set forth in part in the description that follows, and in part will be obvious from the description or may be learned by practice of the aspects described below. The advantages described below will be realized and attained by means of the elements and combinations particularly pointed out in the appended claims. It is to be understood that both the foregoing general description and the following detailed description are example and explanatory only and are not restrictive.

### BRIEF DESCRIPTION OF THE DRAWINGS

The accompanying figures, which are incorporated in and constitute a part of this specification, illustrate several aspects described below.

FIG. 1A-FIG. 1F show diagrams of example thermoformed polymeric devices in accordance with various illustrative embodiments.

FIG. 2A and FIG. 2B show diagrams of example method of fabricating the thermoformed polymeric devices (e.g., of FIG. 1A-FIG. 1E) in accordance with various illustrative embodiments.

FIG. 3 shows a method of operating an expandable conduit device in accordance with an illustrative embodiment.

FIG. 4A-FIG. 4D show a top view and side view of a valved conduit with a 14 mm straight sinus (A, C) and a 14 mm with sinus (B, D).

FIG. 5A-FIG. 5B show flow rate (A) and pressure (B) waveforms for 14 mm sinus conduit.

FIG. 6A-FIG. 6B show an explanted (6 months) surgical valve with HA/LLDPE (A) and hard histology section stained with Sanderson's bone stain (B). The clear leaflet is shown in the bottom of 6B.

FIG. 7A-FIG. 7B show the cytocompatibility of ADSCs (A) and HDFs (B). The asterisk represents statistical differences with their respective treatment groups (P=0.05, n=4).

FIG. 8A-FIG. 8C show SEM images of fixed platelets on HA/LLDPE and controls.

FIG. 9 shows an example PET fabric embedded between LLDPE sheets.

FIG. 10A-FIG. 10J show en face (A) and side view (B & E) pictures of the vacuum-formed clear plastic 26-mm LLDPE valved conduit. FIG. 10C and FIG. 10D show the still images of the same conduit closing and opening under pulsatile flow. FIG. 10F-FIG. 10J are the respective images of the 14-mm conduits.

FIG. 11A-FIG. 11B show flat LLDPE that was thermoformed maintains its shape after HA treatment and perfectly matches the mold dimensions (A). Contact angles on 3D thermoformed leaflets before HA treatment (untreated) and after treatment indicate no difference from contact angles on flat (no thermoforming) leaflets that have been treated with HA (B). Columns sharing the same symbol are not significantly different at p<0.05, n=3.

FIG. 12A-FIG. 12B show the black lines in the PIV velocity field symbolize the conduit's leaflets (A) the flow and transvalvular waveforms and the dashed line represents peak systole where the PIV data was captured (B).

FIG. 13A-FIG. 13C show conduit modeling. A) CT images from patients. B) 3D reconstruction from CT images and cut lines to remove the pulmonary valve for implanting the conduit. C) Conduit implantation between RVOT and PA. PA (pulmonary artery), MPA (main pulmonary artery), RPA (right pulmonary artery), LPA (left pulmonary artery), RVOT (right ventricular outflow tract).

FIG. 14A-FIG. 14I show a workflow of designing a patient-specific valved conduit. 14A shows patient-specific conduit geometry (PSCG) mold. Patient-specific pulmonary artery conduits are made (14a to 14C) in parallel to the valved conduits (14E to 14I). The two processes end with their respective parts combined (14D).

FIG. 15A-FIG. 15C show a method for incorporating PET sewable rings to anastomoses.

FIG. 16A-FIG. 16F shows a schematic of a thermoforming process to form an example valved conduit.

FIG. 17A-FIG. 17E show en face (A) and side view (B, E) pictures of clear plastic LLDPE valved conduit. FIG. 17C and FIG. 17D show images of a conduit under pulsatile flow during peak diastole and systole, respectively.

FIG. 18A-FIG. 18E show a diagram of a right ventricle-pulmonary artery with PET sewable rings.

FIG. 19 shows a perspective view of a conduit device.

FIG. 20 shows a top cross-sectional view of an expandable conduit device.

FIG. 21 shows a perspective view of an expandable conduit device having a longitudinal pleated or folded region.

FIG. 22 shows a perspective view of an expandable conduit device having a circumferential pleated or folded region.

FIG. 23 shows a circumferential review of an expandable conduit device having a circumferential pleated or folded region.

FIG. 24 shows a perspective view of an expandable valved conduit device

FIG. 25 shows a perspective view of an expandable conduit device with an expandable valve

FIG. 26 shows a perspective view of an expandable conduit device with a transcatheter pulmonary valve (TPV) after expansion.

FIG. 27 is a schematic of a physiological right heart simulator for in-vitro hemodynamic testing.

FIG. 28 is an example right ventricle to pulmonary artery conduit.

### DETAILED DESCRIPTION

The following description of the disclosure is provided as an enabling teaching of the disclosure in its best, currently known examples. Many modifications and other examples disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific examples disclosed and that modifications and other examples are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual examples described and illustrated herein has discrete components and features which may be readily separated from or combined with the features of any of the other several examples without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to the arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an,” and “the” include plural referents unless the context clearly dictates otherwise. Thus, for example, reference to “a compound,” “a composition,” or “a disorder” includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’,” where ‘x’ and ‘y’ are numerical values, includes “about ‘x’ to about ‘y’”.

It is to be understood that such a range format is used for convenience and brevity, and, thus, should be interpreted in a flexible manner to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter, or other quantity or characteristic is “about,” “approximate,” or “at or about” whether or not expressly stated to be such. It is understood that where “about,” “approximate,” or “at or about” is used before a quantitative value, the parameter also includes the specific quantitative value itself unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

The term “ion,” as used herein, refers to any molecule, portion of a molecule, the cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, a portion of a molecule, a cluster of molecules, a molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

“Z¹.” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄)alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification, “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkyl alcohol” is used in another, it is not meant to imply that the term “alkyl” does not also refer to specific terms such as “alkyl alcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl,” as used herein, is a non-aromatic carbon-based ring composed of at least three carbon atoms. Examples of cycloalkyl groups include but are not limited to cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acetal” as used herein is represented by the formula (Z¹Z²) C(═OZ³) (═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄)alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Example Device

### Conduit Device

Provided herein is a conduit device including a thermoformed conduit including a first low melting point thermoplastic material suitable for thermoforming, the conduit having a first end and a second end and defining a channel therebetween. The conduit device is configured to be surgically implanted in a subject's heart or blood vessel. In some examples, the conduit device has an annular shape.

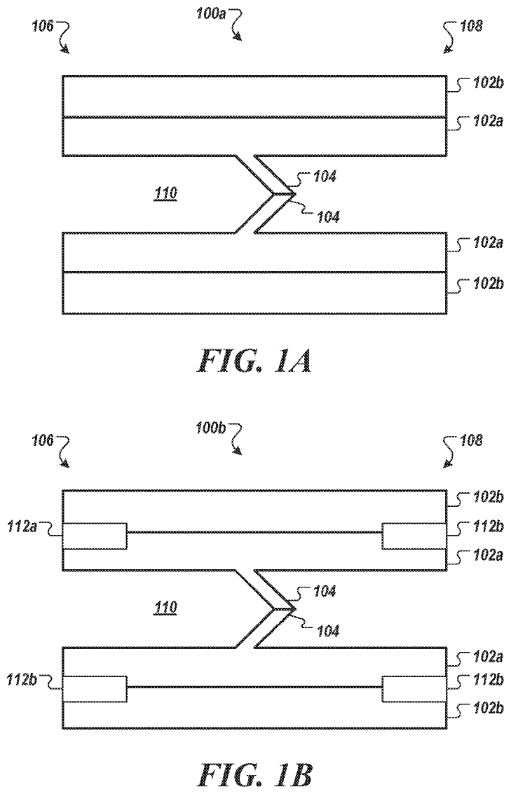

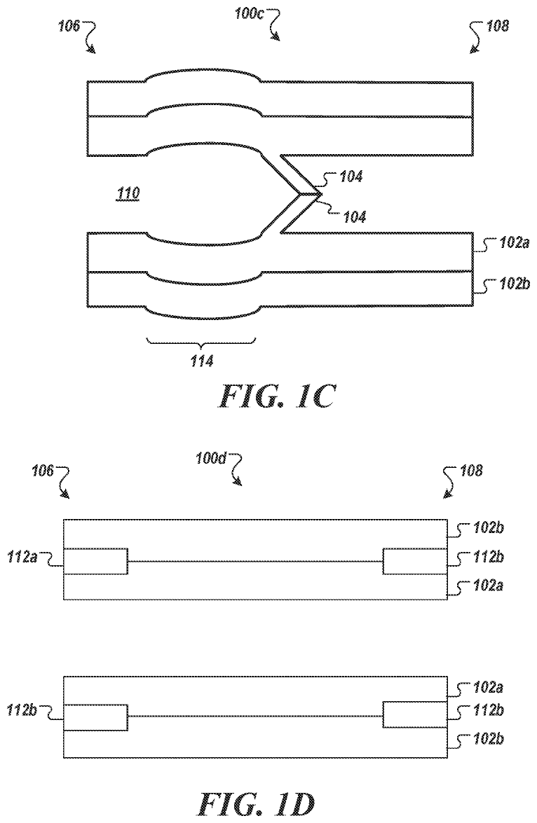

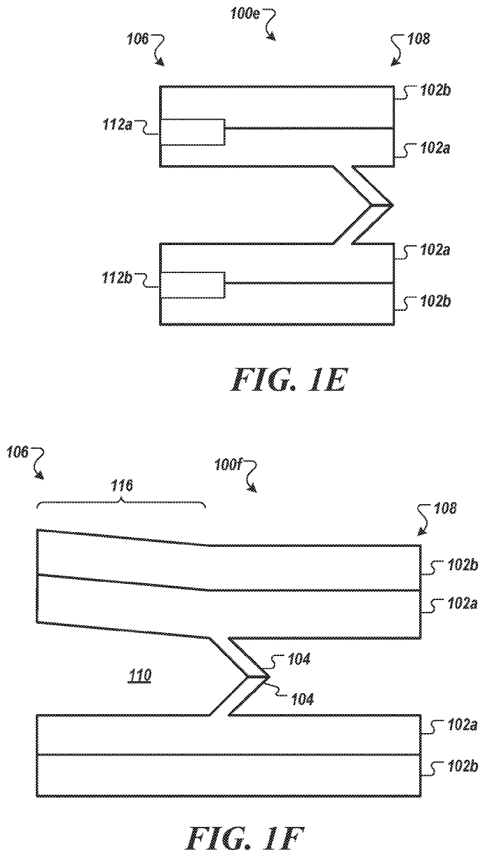

FIG. 1A-FIG. 1F show diagrams of example thermoformed polymeric devices 100 in accordance with various illustrative embodiments.

In the example shown in FIG. 1A, the thermoformed polymeric conduit 100 (shown as valve conduit 100a) for a heart valve formed via a thermoforming process is shown that can be made to be (i) an expandable and resizable conduit that is flexible, deformable, and resizable after being formed or (ii) a non-expandable conduit. The thermoformed conduit 100a includes two or more layers 102 (shown as 102a, 102b) of low melting point thermoplastic material suitable for thermoforming, the inner layer 102a forming a set of two or leaflets 104 therein in which the leaflets form a valve, e.g., that functions based on a pressure differential. The outer layer 102b forms a conduit with the inner layer 102b. The layers are fused or bonded to form a valved conduit. The inner layer 102a preferably has a thinner thickness as compared to the outer layer 102b to provide for the flexibility in the inner layers as the leaflets, and the outer layers being more durable. In some embodiments, the dimensions may reversed, or other layers may be employed.

In FIG. 1A, the thermoformed conduit 100a has a first end 106 and a second end 108 and defines a channel 110 therebetween. The thermoformed conduit 100a is configured to be surgically implanted in a subject's heart or blood vessel. The thermoplastic material may include additives, e.g., hyaluronan or others described herein, in an interpenetrating network to be anti-thrombotic and/or calcification resistant.

A valve is a device for controlling the passage of fluid through a passageway, such as a conduit, by opening, closing, or partially obstructing the passageway. In regard to cardiovascular applications, a valve prevents the backward flow of blood. There are four valves in a human heart: tricuspid valve, pulmonary valve, mitral valve, and aortic valve; any one of which can be replaced or augmented by the exemplary device described herein. In one example, the pulmonary valve has three leaflets and allows blood to pump from the right ventricle to the pulmonary artery. This artery leads to the lungs, where the blood picks up oxygen. The pulmonary valve prevents blood from going backward from the pulmonary artery to the right ventricle. The valve provided herein is a replacement valve for a defective or non-existent pulmonary valve in a subject with CHD. Similar to the human heart, the valve herein, upon administration of the device, prevents backward blood flow from the pulmonary artery to the right ventricle.

FIG. 1B shows the thermoformed polymeric valved conduit 100 (shown as valve conduit 100b) of FIG. 1 in which the conduit 100b is further fabricated with a sewable or sewing ring 112 (shown as 112a, 112b) formed at the first end 106 and the second send 108, respectively. In the example shown in FIG. 1B, the sewable or sewing ring 112 is shown embedded between the inner and the outer layers 102a, 102b. In other embodiments (not shown), the sewable or sewing rings may be positioned and bonded onto the outer layer 102b.

The sewable or sewing ring allows for the device (e.g., 100) to be sutured to its corresponding region in the subject's body in order to position it in an operative position. The sewable or sewing ring(s) may be formed of woven material, a fabric material, the thermoplastic material of the inner and outer layers (e.g., 102a, 102b), another type of thermoplastic material different from that of the inner or outer layers, or another type of material.

FIG. 1C shows another embodiment of the thermoformed polymeric valved conduit 100 (shown as 100c), in which the conduit 100b is designed with a sinus 114. The sinus 114 may be incorporated into the embodiments of FIGS. 1A, 1B, or other embodiments described herein. The sinus 114 is an enlarged opening or hollow structure that widens or enlarges the circumferential profile of the conduit 100c from nearby circumferential positions to allow for faster closing times for valves.

FIG. 1D shows another embodiment of a thermoformed polymeric conduit 100 (shown as a conduit 100d). The thermoformed polymeric conduit 100d may be used, for example, for vascular replacements or augmentation.

FIG. 1E shows another embodiment of the thermoformed polymeric conduit 100 (shown as a transcatheter pulmonary valve 100e), or a part thereof. Various TPV is referenced herein to which the thermoformed polymeric conduit 100e may be embedded or employed therewith.

FIG. 1F shows another embodiment of the thermoformed polymeric conduit 100 (shown as 100f) having asymmetrically circumferential region 116. Indeed, via use a mold, the thermoformed polymeric conduit 100 (including 100f) can be fabricated to have personalized dimension and shape for particular patient. The fabrication may be made based on scanned images or measurements acquired for the patient to which the thermoformed polymeric conduit 100 can be customized.

A thermoplastic, as used herein, is a plastic polymer material that becomes pliable or moldable at a certain elevated temperature and solidifies upon cooling. Thermoplastics include but are not limited to, acrylic, acrylonitrile butadiene styrene, nylon, polylactic acid, polybenzimidazole, polycarbonate, polyether sulfone, polyoxymethylene, polyether ether ketone, polyetherimide, polyethylene, polyphenylene oxide, polyphenylene sulfide, polypropylene, polystyrene, polyvinyl chloride, polyvinylidene fluoride, and polytetrafluoroethylene.

Thermoforming can use both vacuum and air pressure to push and/or pull a soft, heated thermoplastic against a custom mold or tool. Upon being manipulated to its desired shape, the thermoplastic is cooled.

Molds for thermoforming include molds comprising plaster of Paris, wood, plastic, aluminum, or any combination thereof. The mold is designed such that it has the shape of the desired shape for the thermoplastic. These molds can also be negative molds in the case of hollow parts.

In some examples, the conduit device includes an inner layer that is made of the first low melting point thermoplastic material and an outer layer disposed over the first sewable ring, the second sewable ring, and the conduit such that the first and second sewable rings are fixed between the conduit and the outer layer. In some examples, the outer layer comprises a third low melting point thermoplastic material.

In some examples, the conduit device further comprises a hyaluronan, such as a hyaluronic acid additive. Hyaluronan, also referred to as hyaluronic acid (HA), is an anionic, nonsulfated glycosaminoglycan distributed throughout connective, epithelial, and neural tissues. HA has the following formula:

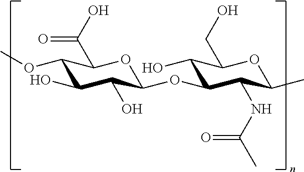

Hyaluronanis a polymer of disaccharides, which are composed of D-glucuronic acid and N-acetyl-D-glucosamine, linked via alternating β-(1→4) and β-(1→3) glycosidic bonds. Hyaluronic acid can be 25,000 disaccharide repeats in length. Polymers of hyaluronic acid can range in size from 5,000 to 20,000,000 Da in vivo. In some examples, HA is added to the first low melting point thermoplastic material.

In further examples, at least one of the first thermoplastic material, the second thermoplastic material, or the third thermoplastic material comprises a polyethylene. Polyethylene is a member of the family of polyolefin resins and is a synthetic resin made from the polymerization of ethylene. Ethylene (C₂H₄) is a gaseous hydrocarbon commonly produced by the cracking of ethane. Ethylene molecules comprise two methylene units linked together by a double bond between the carbon atoms. Upon polymerization, the double bond is broken and the resultant extra single bond is used to link to a carbon atom in another ethylene molecule, thereby making a large, polymeric (multiple-unit) molecule with a repeating ethylene unit.

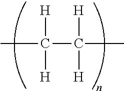

In specific examples, the polyethylene comprises linear low-density polyethylene (LLDPE). LLDPE is a substantially linear polyethylene polymer with significant numbers of short branches and can be made by copolymerization of ethylene with longer-chain olefins. LLDPE differs structurally from low-density polyethylene (LDPE) because of the absence of long-chain branching. LLDPE is generally produced at lower temperatures and pressures by copolymerization of ethylene.

Production of LLDPE is initiated by transition metal catalysts, which can include but is not limited to Ziegler or Phillips types of catalysts. The polymerization process can be done either in the solution phase or in gas phase reactors. LLDPE has a higher tensile strength, higher impact, and high puncture resistance than LDPE. It is also flexible and elongates under stress.

In certain examples, the second thermoplastic material comprises polyethylene terephthalate (PET). PET is a thermoplastic polymer resin and comprises repeating C₁₀H₈O₄ units. PET can be amorphous or semi-crystalline. PET can be made using bis(2-hydroxyethyl) terephthalate. Polymerization can occur through a polycondensation reaction of the monomers immediately after esterification/transesterification with water as the byproduct.

In some examples, the hyaluronan and LLDPE are arranged in an interpenetrating network. An interpenetrating network comprises two or more networks that are at least partially interlaced on a polymer scale but not covalently bonded to each other. In some examples, the network cannot be separated unless chemical bonds are broken. The networks can be entangled in such a way that they are concatenated and cannot be pulled apart but bonded to each other by any chemical bond. Interpenetrating networks can also include semi-interpenetrating polymer networks and pseudo-interpenetrating polymer networks. An interpenetrating network can broaden the glass transition region in comparison to the component polymers. This increased glass transition region can provide increased mechanical damping properties over a wide range of temperatures and frequencies due to a relatively constant and high phase angle.

In some examples, at least one of the first thermoplastic material, the second thermoplastic material, or the third thermoplastic material is a woven material. In some examples, at least one of the first thermoplastic materials, the second thermoplastic material, or the third thermoplastic material, is a non-woven material.

In some examples, the conduit device is calcification resistant. Calcification occurs when there is a buildup of excess calcium on the device. Calcification can cause the failure of contemporary bioprosthetic heart valves and can limit the functional lifetime of experimental and clinical polymeric heart valves. The components of and method of making the device, as discussed herein, in some examples, result in a calcification-resistant conduit device, which helps to prevent failure of the device and lengthens the lifetime of the device.

In some examples, the conduit device is anti-thrombotic. Thrombosis refers to the build-up of blood clots, which can cause problems in the use of medical devices in a subject. The components of and method of making the device, as discussed herein, in some examples, result in an anti-thrombotic conduit device, which prevents the formation of blood clots on the device.

In some examples, the conduit is deformable to be resizable. Deformable is used herein to mean the ability to become misshapen or distorted or changed in shape to change the size of the conduit. Resizable is used herein to mean the ability to change in size due to plastic deformation (will not return to its original shape). The resizing can be along the longitudinal length of the device or circumferentially for the diameter of the device. The device may include foldable or pleated geometric structures that are designed and fabricated into the device to facilitate the resizing.

In some examples, the conduit is deformable such that it is expandable from a first-installed configuration to a second-modified configuration. When expanded from the installed configuration to the modified configuration, the cross-sectional area of the conduit increases from a first cross-sectional area to an expanded second cross-sectional area. In some examples, this applies to flexibility in administration and/or expandability of the conduit so as to accommodate the growth of the subject, which can be accomplished by tools that include but are not limited to an inflatable member. The use of LLDPE renders the conduit deformable and resizable, which can (i) provide for the ability for the device to be resized to account for somatic growth during the subject's lifetime and/or (ii) provide for more flexibility in administration and also contributes to the personalized nature of the device. CHD can cause differences in the shape and characteristics of the hearts of different subjects and with a deformable conduit, those differences can be accounted for, thereby rendering a device better suited for each subject.

In some examples, the conduit is configured to operate in a non-expandable state. In such examples, the conduit is not configured to expand.

In some examples, the conduit is configured to operate in a non-expanded state. In such examples, the conduit is operable in a first installed configuration and may optionally be expanded to a second modified configuration having an expanded cross-sectional area thereafter.

In further examples, a reinforcing structure is disposed between the conduit and the outer layer. The reinforcing structure, as used herein, could be a guiding, reinforcing, and/or a sliding/locking mechanism to allow for resizing of the device in both diameter and length.

In some examples, the reinforcing structure is a coil. In further examples, the reinforcing structure is a stent-like structure comprising at least one strut.

In specific examples, the reinforcing coil comprises metal. In further examples, metal can include, but is not limited to, nitinol, Co—Cr, stainless steel, or any combination thereof.

In certain examples, the reinforcing coil comprises a polymer. In further examples, the polymer can include but is not limited to, nylon, polyethylene, polyester, polytetrafluoroethylene (PTFE), or epoxy.

In some examples, the valve is expandable and is not connected to the conduit or the outer layer. Expandable, as used herein, refers to the valve's ability to increase its length and annulus diameter of the device upon expansion of the device by a transcatheter balloon dilator.

In specific examples, the device comprises a transcatheter pulmonary valve (TPV), wherein the TPV is positioned inside the conduit and between the leaflets upon separation of the leaflets after expansion. A transcatheter pulmonary valve (TPV) is a replacement pulmonary valve that is inserted via a catheter.

Conduit Device or Valved Device with Expandable Structure

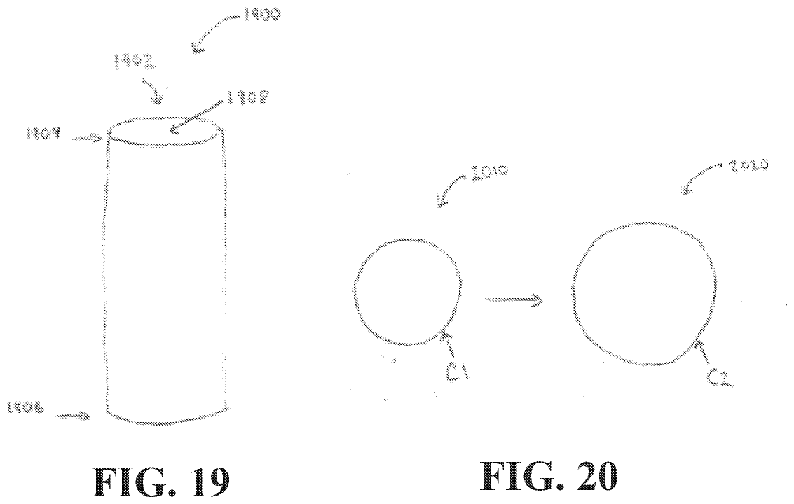

An example expandable conduit device 100 (shown referenced as 1900) is illustrated in FIG. 19 and includes an expandable thermoformed conduit 1902 formed of a first low melting point thermoplastic material suitable for thermoforming. The thermoformed conduit includes a first end 1904 and a second end 1906 and defines a channel 1908 therebetween. The expandable conduit device 1900 is configured to be surgically implanted in a subject's heart or blood vessel, wherein the channel 1908 is configured to permit ingress and egress of blood.

In some examples, the expandable thermoformed conduit 1902 is configured to expand to the second circumferential profile C2 via plastic deformation when mechanically induced by an expandable device.

In some examples, the expandable thermoformed conduit 2102 includes a circumferential pleated or folded region 2108 at a portion 2110 of the first circumferential profile 2106 when in the first configuration 2010, the circumferential pleated or folded region 2110 extending along the longitudinal profile or a portion thereof.

In further examples, the expandable conduit device 2200 includes a thermoformed conduit 2202 having a length defined by a longitudinal profile 2204 and includes a longitudinal pleated or folded region 2206 along the longitudinal profile 2204 or a portion 2208 thereof.

In some examples, the expandable thermoformed conduit includes both a circumferential pleated or folded region and a longitudinal pleated or folded region.

In further examples, the expandable conduit device 2400 comprises a reinforcing structure 2402 disposed within the thermoformed conduit 2404.

In some examples, the reinforcing structure 2402 includes a stent-shaped structure comprising at least one strut.

In further examples, the reinforcing structure 2402 includes a metallic composite or alloy.

In some examples, the reinforcing structure 2402 includes a coil-shaped structure.

In further examples, the reinforcing structure 2402 includes a polymer or a thermoplastic material.

In some examples, the expandable conduit device comprises two or more leaflets configured to operate as a valve by moving collectively between an open state and a closed state, the two or more leaflets each comprising a first end and a second end and defining a curved length therebetween, at least one of the two or more leaflets being sized to (i) form the closed state in connection with other leaflets in the first configuration and (ii) form the closed state in connection with other leaflets in the second configuration.

In further examples, the expandable conduit device further comprises any of the features as described herein.

### Example Method of Fabrication of the Thermoformed Polymetric Conduit Device

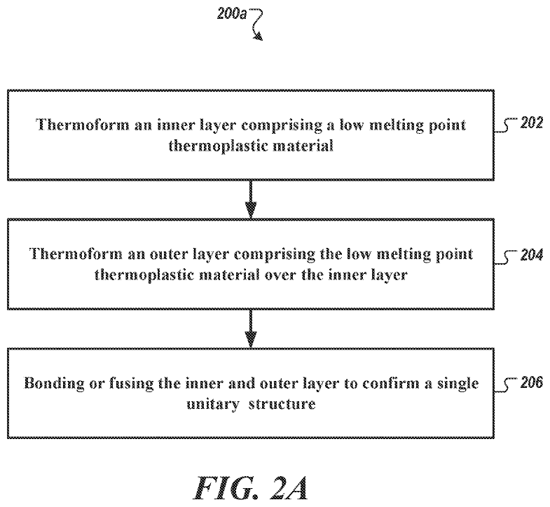

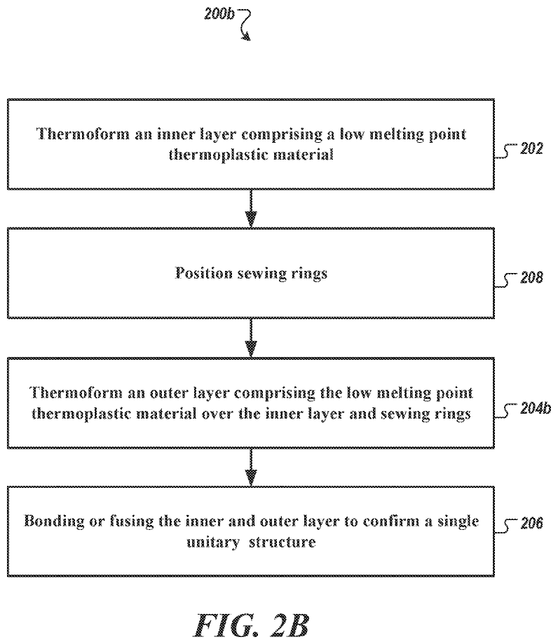

FIG. 2A and FIG. 2B show diagrams of example methods 200 (shown as 200a and 200b) of fabricating the thermoformed polymeric device (e.g., of FIG. 1A-FIG. 1E) in accordance with various illustrative embodiments.

In the example shown in FIG. 2A, the method 200a of fabricating the conduit device (e.g., 100a, 100b, 100c, 100d, 100e, 100f, etc.) includes thermoforming 202a a thermoplastic to form an inner layer (e.g., 102a) of the conduit via a thermoforming process that conforms the conduit to a first mold. Thermoforming the thermoplastic may include preheating the thermoplastic with the mold. Method 200a then includes thermoforming 102b another thermoplastic (e.g., the same type as 202) to form another outer layer (e.g., 102b) of the conduit via a thermoforming process that conforms the conduit to the first mold or another mold. Thermoforming the second thermoplastic may include preheating the second thermoplastic with the second mold mounted on the first mold, thermoforming the second thermoplastic on the second mold to form the outer layer, and bonding the conduit and the outer layer to fuse the thermoformed conduit and thermoformed outer layer together. Method 200a then includes fusing or bonding the inner layer and the outer layer together.

FIG. 2B shows another method 200b of fabricating the thermoformed polymeric device with sewing rings (e.g., 112). Method 200b includes thermoforming 202a a thermoplastic to form an inner layer (e.g., 102a) of the conduit via a thermoforming process that conforms the conduit to a first mold. Method 200b then includes positioning a first and second sewable rings (e.g., 112) within structures of the thermoformed conduit and thermoforming 204 (shown as 204b) the second thermoplastic into the outer layer (102b) that conforms the second thermoplastic to the first mold or another mold. Thermoforming the second thermoplastic includes preheating the second thermoplastic with the second mold mounted on the first mold, thermoforming the second thermoplastic on the second mold to form the outer layer, and bonding the conduit and the outer layer to fuse the thermoformed conduit and thermoformed outer layer together. In some examples, bonding the inner and outer layers includes applying heat to the inner and outer layers.

As illustrated in FIG. 14E-FIG. 14I, in some examples of forming a valved conduit, the method of fabricating the conduit device includes thermoforming a first thermoplastic to form the leaflets via a thermoforming process that conforms the first thermoplastic to a first mold and thermoforming a second thermoplastic to form a thermoformed conduit via a thermoforming process that conforms the second thermoplastic to a second mold. The first mold has a generally cylindrical body and includes a centrally disposed peak structure with angled or curved sides. In such examples, thermoforming the first thermoplastic on the first mold produces arched or angled leaflets corresponding to the peak structure of the first mold. Then, a second mold, having a generally cylindrical body similar to the first mold and configured to compliment the first mold, is disposed complimentary to the first mold such that the peak structure of the first mold is received into a depression in the second mold. That is, the second mold is disposed over the arched or angled leaflets formed by the first thermoplastic. The second thermoplastic is then thermoformed to the second mold to form a thermoformed conduit. In such examples, the second thermoplastic is disposed about an outer surface of both the first and second molds such that it contacts the first thermoplastic. The first and second thermoplastics are then bonded to fuse the two layers such that the second thermoplastic forms a generally cylindrical outer layer conduit, and the first thermoplastic forms an inner layer that includes leaflets. In some examples, bonding the first and second thermoplastics includes heating the first and second thermoplastics to a temperature sufficient to fuse the two layers.

In some examples, the leaflets are shaped to be independently movable by the first mold, while in other examples, the leaflets are formed in a single molded formation that must be cut or formed into a desired shape such that the leaflets are able to move relative to one another.

The second mold may include an outwardly projecting bulbous region. When the second thermoplastic is thermoformed on the second mold and is disposed over the bulbous region, the second thermoplastic forms a sinus.

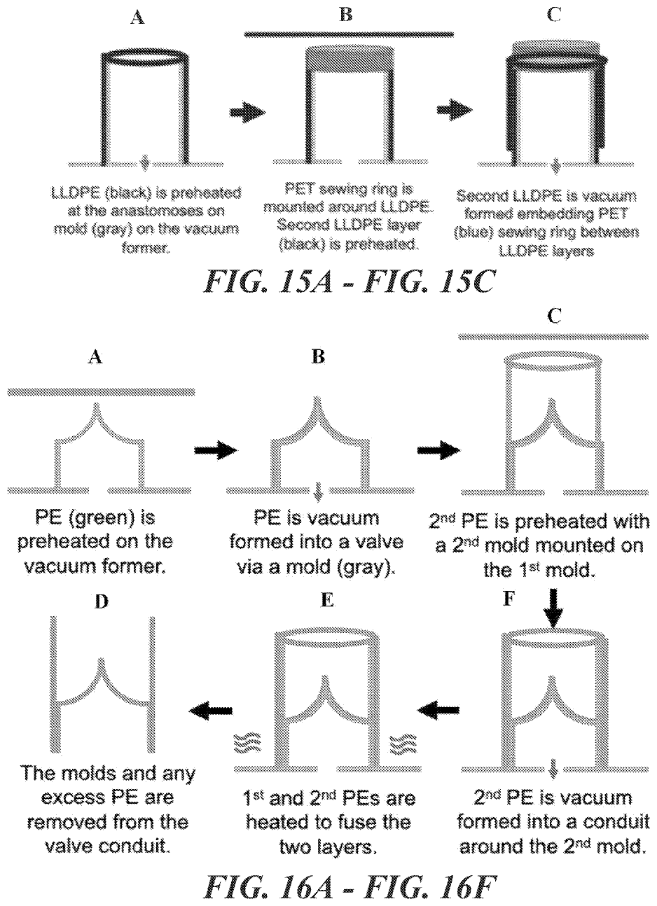

FIG. 15A-FIG. 15C show an example process of thermoforming the conduit device comprising sewable rings, wherein LLDPE is thermoformed on a first mold to form a thermoformed conduit (FIG. 15A), a first and second sewable rings are mounted on the thermoformed conduit and a second layer of LLDPE is thermoformed on a second mold to form an outer layer wherein the first and second sewable rings are positioned between the thermoformed conduit and the outer layer (FIG. 15B), and the outer layer is vacuum formed to the thermoformed conduit so as to embed the first and second sewable rings between the conduit and outer layer (FIG. 15C). In some examples, the method of fabricating the conduit device includes thermoforming a thermoformed conduit, where the thermoformed conduit includes a first low melting point thermoplastic material suitable for thermoforming. The thermoformed conduit also includes a first end and a second end and defines a channel therebetween. The conduit device is configured to be surgically implanted in a subject's heart or blood vessel. The thermoformed conduit may further correspond to any of the examples previously described.

In some examples, the method of fabricating the conduit device includes thermoforming a thermoformed conduit, where the thermoformed conduit includes an inner layer. In such examples, the inner layer includes a first low melting point thermoplastic material and a second low melting point thermoplastic material, each suitable for thermoforming. The thermoformed conduit also includes a first end and a second end and defines a channel therebetween. The conduit device is configured to be surgically implanted in a subject's heart or blood vessel. The thermoformed conduit may further correspond to any of the examples previously described.

In some examples, preheating of the thermoplastic occurs on a vacuum former. In further examples, thermoforming can include vacuum-induced thermoforming, pressure-induced thermoforming, or a combination thereof.

In some examples, the second mold comprises a sinus such that the outer layer formed from the third thermoplastic comprises the sinus.

In some examples, the method comprises positioning a first sewable ring and a second sewable ring within structures of the thermoformed conduit, wherein the thermoformed conduit device further comprises an outer layer, wherein the thermoforming embeds the first sewable ring and the second sewable ring within the thermoformed conduit and fuses the inner layer and the outer layer with the first sewable ring and a second sewable ring embedded therebetween.

In use, the first and second sewable rings provides a region at which the conduit device can be coupled to the anatomy of a subject, such as by sewing.

In some examples, the conduit device may not include leaflets or another valve structure. In some examples, the sewable rings on conduit devices having leaflets or other valve structures may be used to couple said conduit devices to conduit devices without leaflets or other valve structures. In some examples, as illustrated in FIG. 14D, conduit devices having leaflets or other valve structures may be coupled to conduit devices without leaflets or other valve structures via stents. In such examples, welding methods may also be used to further facilitate the coupling.

In some examples, the method further includes fabricating a personalized conduit specific to a subject in need thereof, wherein fabricating the personalized conduit comprises receiving a topology image or scan of the subject's anatomy. For example, the subject's anatomy may include the right ventricle of the heart and the pulmonary artery. The topology image or scan may be obtained using various techniques, such as MRI, CT, or ultrasound. The method further includes obtaining a topology mapping of the subject's anatomy from the topology image or scan and fabricating one or more molds based on the topology mapping. Thereafter, the method further comprises thermoforming the thermoformed conduit over the fabricated one or more molds.

In specific examples, the step of obtaining a topology mapping includes determining at least one of a curvature, a diameter, and a length that are used to create the one or more molds. The curvature, a diameter, and a length correspond to a portion of the thermoformed conduit to be thermoformed using the one or more molds.

In some examples, the steps are performed for any one of the devices disclosed herein.

### Example Method of Using the Thermoformed Polymeric Device as an Expandable Device

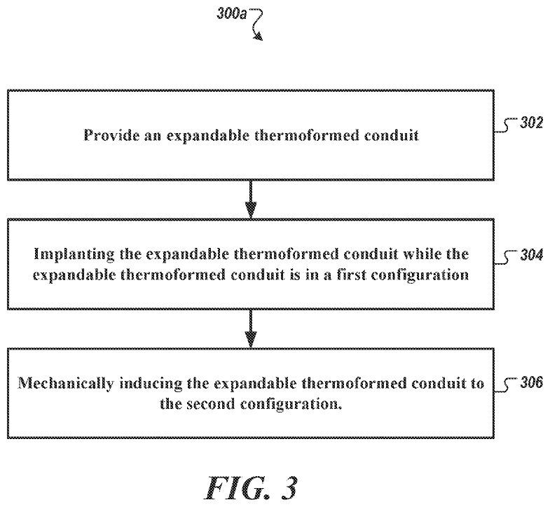

FIG. 3 shows a method 300 of operating an expandable conduit device in accordance with an illustrative embodiment. Method 300 comprises providing 302 an expandable thermoformed conduit comprising a low melting point thermoplastic material suitable for thermoforming. The thermoformed conduit may include a first end and a second end and define a channel therebetween, wherein the conduit device is configured to be surgically embedded into a subject's heart. The expandable thermoformed conduit is configured to have a first configuration having a first circumferential profile at a first position in the channel and a second configuration having a second circumferential profile at the first position, wherein the second circumferential profile has a larger cross-sectional area than the first circumferential profile; implanting 304 the expandable thermoformed conduit while the expandable thermoformed conduit is in the first configuration; and mechanically inducing 306 the expandable thermoformed conduit to the second configuration.

In some examples, mechanically inducing comprises positioning an expandable member in a position within the channel of the expandable thermoformed conduit; and expanding the expandable member to adjust the cross-sectional area of the expandable thermoformed conduit at the first position to the second circumferential profile.

In further examples, the expandable thermoformed conduit is configured as a transcatheter pulmonary valve.

In some examples, the expandable thermoformed conduit is configured as a right ventricle to pulmonary artery conduit.

In further examples, the first configuration is sized for a child or a young adult.

In some examples, the second configuration is sized for a young adult or an adult.

In further examples, the steps of this method are performed on the expandable conduit device as disclosed herein.

Example Method of Expanding an Expandable Member. Further provided herein is a method of expanding an expandable member in proximity to the expandable conduit device provided herein comprising, positioning the expandable member in proximity to the expandable conduit device; deforming the expandable member such that it contacts an inner wall of the expandable thermoformed conduit and exerts an outwardly directed radial force thereupon. In response, the expandable thermoformed conduit is deformed into a plastic deformation region such that it transitions to an expanded configuration compared to an original expansion; and maintains the expanded configuration.

Transcatheter Pulmonary Valve. In some examples, the method further comprises positioning a TPV in proximity to the expandable conduit device after deforming the expandable member and deploying the TPV within the conduit device.

A number of examples of the disclosure have been described. Nevertheless, it will be understood that various modifications may be made without departing from the spirit and scope of the invention. Accordingly, other examples are within the scope of the following claims.

By way of non-limiting illustration, examples of certain examples of the present disclosure are given below.

### EXAMPLES

The following examples are set forth below to illustrate the methods and results according to the disclosed subject matter. These examples are not intended to be inclusive of all aspects of the subject matter disclosed herein but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Additional Example 1: Effect of Bulging Sinus on Hemodynamic Performance of Polymeric Rv-Pa Conduit for Pediatric and Adult Patients

### Introduction

Congenital heart defects (CHD) are among the most common birth defects in the United States and a leading contributor to infant mortality rates across the globe. In 2017, CHD caused about 181,000 deaths in infants, accounting for 70% of the total CHD mortality rate worldwide¹. A right ventricle to pulmonary artery (RV-PA) conduit is often used in procedures for various congenital heart defects such as pulmonary atresia, Tetralogy of Fallot, truncus arteriosus, and right ventricle outflow tract reconstruction. This device helps restore the pulmonary blood flow from the heart. Currently, available conduit options include homografts, decellularized allografts, commercial xenografts, prostheses made from expanded polytetrafluoroethylene (ePTFE) and/or polyethylene terephthalate (PET) that are hand sewn intraoperatively, and a newly developed tissue engineering conduit, the Xeltis pulmonary valve (Corno, 2004; Ootaki, 2018; Yamamoto, 2020; Proden, 2021; Carreon, 2019). The challenges with these existing conduits include thrombus formation, calcification, stenosis, regurgitation, and sternal compression, which affects the device's longevity leading to surgical reinterventions (Prawel, 2014). Therefore, there is a critical need to investigate biocompatible materials, optimize the design for these conduits, and introduce geometrical modifications that can alleviate these issues. Our group has demonstrated the use of a novel biomaterial made of an interpenetrating polymeric network of hyaluronan (HA) and linear low-density polyethylene (LLDPE) for cardiovascular devices, owing to its biocompatibility, anti-calcific and anti-thrombotic properties (Prawel, 2014; Heitkemper, 2018). In addition to these advantages, HA-LLDPE has good hemodynamic performance due to low bending stiffness, high tear and tensile strengths, as well as excellent handling characteristics, and is inexpensive. Therefore, the objective of this pilot project is to demonstrate the potential of the HA-LLDPE conduit for pediatric and adult populations to address many of the drawbacks of the available options. We will look at HA-LLDPE conduit as an alternative to existing options and understand the difference in hemodynamics post-geometrical modifications. Particularly, we will investigate the introduction of a bulging sinus and its effect on the conduit's performance characteristics.

### Methods of Additional Example 1

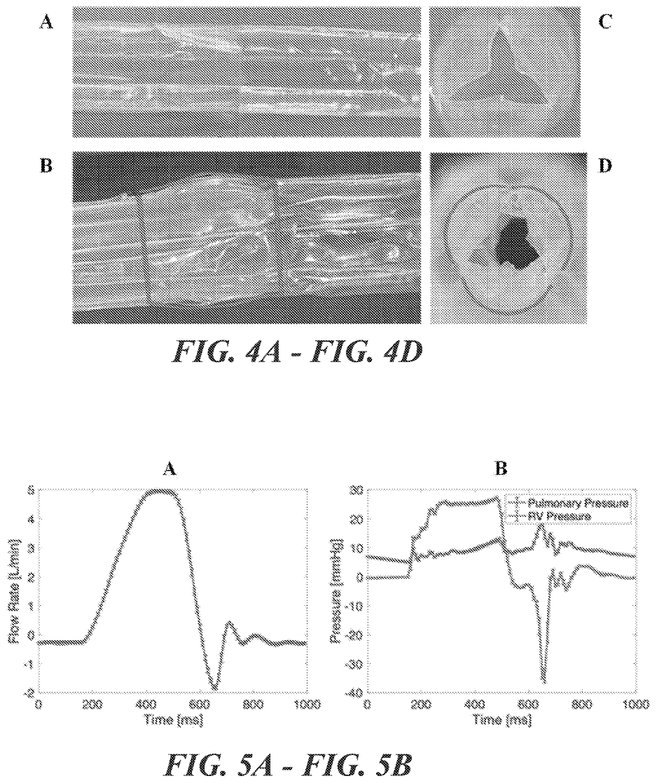

FIG. 4A-FIG. 4D an exemplary valved conduit (corresponding to the device 100 of FIG. 1A. In FIG. 4A, the top view and side view of the valved conduit are shown with a 14 mm straight sinus (subpanels A and C) and a 14 mm with sinus (subpanels B and D).

### Conduit Design

LLDPE conduits of diameters 14 mm and 26 mm were designed with and without a sinus, having the same leaflet geometry as shown in FIG. 4A-FIG. 4D. In order to incorporate the sinus geometry, the correlation between the body surface area (BSA) and sinus of Valsalva, which is 26.79 6.59 mm/m² (r−0.886, p<0.001) when BSA is between 0.5 and 1.0 m², was used (Paytoncu, 2019). Equation 1 gives the relationship between the main pulmonary artery (MPA) and body surface area (BSA) (Sfyridis, 2011) per Equation 1.

$$M P A = ( 1.653 ) ( B S A0 \text{.5} ) + 0.033 M P A = 1.653 B S A0 \text{.5} + 0.033 \qquad ( Eq . 1 )$$

Based on the above relationship, the sinus diameter for each of the designs was calculated and incorporated into their respective designs.

### Valve Fabrication and Conduit Assembly

LLDPE (80 μm thick) sheet was mounted on a vacuum former, preheated for 30 seconds, and vacuum formed into a valve using a 3D printed mold. The process was repeated with another LLDPE sheet (e.g., 80-120 μm thick) thermoformed around a tubular mold that it mounted on the valve, which created the conduit. Further heating of the valved conduit along its wall fused two LLDPE layers together. In order to incorporate a sinus geometry into the valve, the molds used to create the valved conduits will be altered. FIG. 4A-FIG. 4D show the two conduit designs after thermoforming.

Treatment of ePTFE Vascular Grafts with Hyaluronic Acid

Sections measuring 10 cm were cut from an ePTFE tube and cleaned by submersion in ethanol for at least 1 hour. After cleaning, the sections were dried under a vacuum (−25 inHg) for at least 12 hours prior to treatment with hyaluronan-cetyltrimethylammonium.

Hyaluronan-cetyltrimethylammonium was synthesized by mixing a 0.3% (w/v) solution of sodium hyaluronate in deionized water with a 1.0% solution of cetyltrimethylammonium bromide deionized water. The precipitate, hyaluronan-cetyltrimethylammonium (HACTA), was collected, washed with water, and dried for at least 48 hours at −25 inHg and 40-50° C.

A 1.0% (w/v) HACTA solution was prepared by dissolving HACTA in 200-proof ethanol for 7 hours. The 10 cm sections of ePTFE were submerged in 70 ml of the 1.0% w/v ethanol solutions at 55° C. for two hours. After, the ePTFE sections were slowly withdrawn from the solution at approximately 3.5 cm/hr at 50° C. and under a vacuum pressure of −15 inHg. After withdrawal from the solution, the ePTFE sections were dried for 1.5 hours at 50° C. and −25 inHg prior to crosslinking.

A 10% (v/v) of toluene diisocyanate and xylenes was prepared and heated to 65° C. Each ePTFE conduit coated in HACTA was placed in a jar above 10 ml of the toluene diisocyanate and xylene solution for 1 hour to allow the toluene diisocyanate to crosslink the HACTA coating the ePTFE. After 1 hour, the samples were removed and dried overnight under a −25 inHg vacuum.

The HACTA coating was sonicated in 0.2 M NaCl in DI/ethanol (1:1, v/v) solution for one hour. Afterward, the solution was replaced, and sonication was repeated twice more for a total of 3 sonication regimens. Samples were rinsed with DI and then sonicated in 0.2 M NaCl in DI for one hour. Excess CTA was leeched from the material in a 3:2 solution of DI/ethanol for 2 hours. The HA-coated ePTFE conduit underwent a final sonication in DI for 30 minutes before being dried overnight under vacuum (−25 inHg).

### Patency Assessment of the HA-ePTFE Conduit

Prior to implantation as a Blalock-Taussig shunt, the HA-ePTFE, the conduit was stored in 70% ethanol.

Only one HA-ePTFE conduit and one untreated ePTFE conduit was implanted at a Blalock-Taussig shunt. After 3 months, the conduits were explanted, and the HA-ePTFE conduit had maintained patency. Upon visual inspection, the HA-ePTFE conduit had noticeably less tissue growth and lesions both internally and externally.

### In Vitro Hemodynamic Assessment

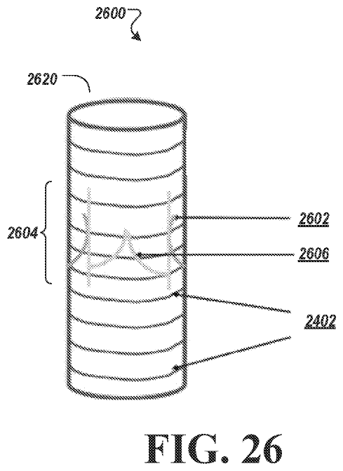

LLDPE valved conduits, with and without a sinus of Valsalva, of two diameters (14 and 26 mm) were fabricated and mounted in an in-vitro model as shown in FIG. 26 and tested under pulsatile flow conditions (8/25 mm Hg diastolic/peak systolic pressure). The 14 mm and 26 mm conduits were tested at pediatric and adult conditions, respectively. A working fluid of 60/40 water to glycerin (99% pure glycerin) was used as a blood analog to provide the required density and kinematic viscosity of 1060 kg/m³ and 3.5×10-6 m²/s, respectively. Pressure gradient and flow waveform were collected. From this data, mean transvalvular pressure gradient (ΔP), peak transvalvular pressure gradient (peak ΔP), effective orifice area (EOA), and a regurgitant fraction (RF) were computed for each of the valved conduits.

### Results of Additional Example 1

The ΔP, peak ΔP, and RF for the 14 mm and 26 mm straight conduits without a sinus are shown in Table 1. The EOA for all samples is close to the minimum device performance required value for the appropriate valve size, between 1.45 cm² for 26 mm ID and approximately 0.47 cm² for 14 mm inner diameter (ID) as extrapolated from the ISO 5840. Additional data for the 14 mm and 26 mm conduits with bulging sinus is being generated for the presentation.

TABLE 1
Hemodynamic data and in-vitro setting
of the LLDPE pulmonary valved conduit.

| | | | |
|---|---|---|---|
| | ΔP | Peak ΔP | (RF) |
| Size | (mmHg) | (mmHg) | (%) |

| (mm) | mean | std | mean | std | mean | std |
|---|---|---|---|---|---|---|
| 14 | 13.6 | ±0.72 | 24.9 | ±1.17 | 2.83 | ±0.74 |
| 26 | 18.1 | ±0.25 | 24.7 | ±0.28 | 9.53 | ±0.49 |

### Discussion

The exemplary HA-LLDPE valved conduits can be employed as a biocompatible conduits that can provide excellent hemodynamics and durability. The pressure gradients of the LLDPE conduits can be comparable to that of Contegra bovine jugular conduits (Sfyridis, 2011). The incorporation of a bulging sinus can improve the overall hemodynamic performance characteristics of the conduit, increasing the longevity of the device. Thus, the development of this conduit would help in the treatment of various complex congenital heart defects.

### Additional Example 2: Design and In Vitro Hemodynamic Assessment of Polymeric RV-PA Conduits

### Introduction

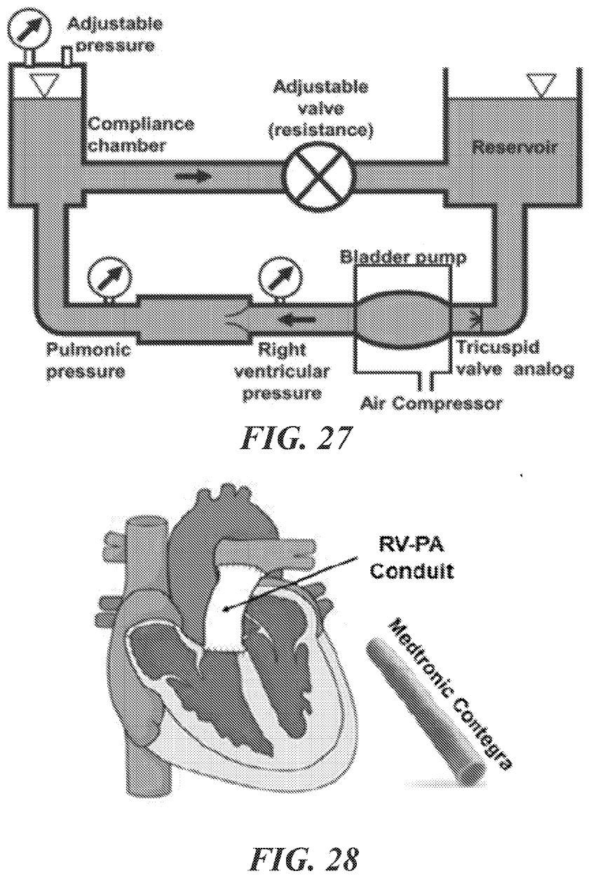

Right ventricle to pulmonary artery (RV-PA) conduits re used in surgical procedures to restore the pulmonary blood flow from the heart (FIG. 27). Drawbacks with currently available conduits include calcification, thrombosis, stenosis, and unavailability in different sizes and shapes (Carreon, 2019). Described herein is a novel biomaterial of an interpenetrating network of hyaluronan (HA) and linear low-density polyethylene (LLDPE) (Formula I), which proved advantageous due to biocompatibility and anti-calcific and anti-thrombotic properties (Prawel, 2014). The use of an HA-LLDPE conduit as an alternative to existing options is demonstrated herein.

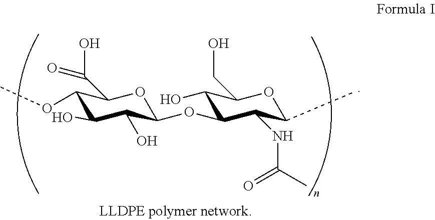

### Methods

LLDPE sheets (80 μm thickness) were thermoformed (FIG. 14E to FIG. 14I) to assemble the conduit and leaflets of different geometries (FIG. 4A to FIG. 4D). Prototypes were tested at pulsatile flow conditions (8/25 mm Hg diastolic/peak systolic pressure) using 60/40 water/glycerin mixture.

### Results and Discussion

The hemodynamics characteristics measured are shown in FIG. 5A, FIG. 5B, and Table 2. The effective orifice area (EOA) for straight conduits within ISO 5840 standard for respective valve diameter.

TABLE 2
Hemodynamic characteristics for polymeric valved conduits.

| | Cardiac output | | | |
|---|---|---|---|---|
| Size (mm) | (L/min) | ΔP (mmHg) | RF (%) | EOA (cm²) |
| 14 (straight) | 1.5 | 13.6 | 2.83 | 0.46 |
| 14 (sinus) | 1.5 | 13.5 | 13.45 | 0.325 |
| 26 (straight) | 5 | 18.1 | 9.53 | 1.33 |

### Summary

Exemplary HA-LLDPE valved conduits provide positive steps towards developing biocompatible conduits with good hemodynamics.

### Additional Example 3: Right Ventricle-to-Pulmonary Artery Valved Conduit

### Background

According to the CDC, congenital heart disease (or defect) (CHD) occurs in about 1% of births in the United States (40,000 births), and about 7,200 CHDs require surgery within the first year of life. In 2010 an estimated one million children and 1.4 million adults were living with congenital heart disease in the US (CDC, 2020). In the US, from 2000-2011, over 20,000 adults with tetralogy of Fallot were admitted to the hospital and the number of pulmonary valve replacements (PVR) increased from 6.8% to 11.3%. The PVR rate doubled over the course of the study, and because percutaneous PVR replacement had no billing code at the time, a significant number of PVR replacements were not counted in the study. PVR was strongly correlated with an increase in the cost of admissions (Stefanescu, 2016). While tetralogy of Fallot is one of the most dangerous CHDs requiring RV-PA conduit implantation, many other CHDs require these conduits for patient survival, including hypoplastic left heart syndrome, pulmonary atresia, truncus arteriosus, congenital aortic stenosis, and transposition of the great arteries (Brown, 2011; Mastropietro, 2019; Brown, 2011; Elder, 2013; Yasukawa; 2020). The highly complex and patient-specific nature of the reconstruction demands durable and personalized designs for RV-PA valved conduits that were custom-made for each patient according to pre-procedural computational planning. Though complete data on RV-PA conduit implants in the United States is elusive, many patients with critical CHD survived into adulthood due to the advancement of pediatric cardiothoracic surgery. These patients will need at least one, if not multiple RV-PA conduits, to survive. These individuals stand to benefit from personalized RV-PA conduits that have long-term patency and address the many shortcomings of current options.

RV-PA conduits used in RVOT reconstruction were vital to providing pediatric patients with various critical CHDs a chance to survive into adulthood (da Costa, 2017; DiBardino, 2014; Kan, 2018; Shinkawa, 2010). Adult patients with heart defects or diseased valves can require an RV-PA conduit, and adults with previously implanted RV-PA conduits often require reintervention due to the limitations of currently available materials (Yuan, 2008; Shinkawa, 2010; Stelzer, 2011). Despite the known challenges such as endocarditis, calcification, thrombus, stenosis, regurgitation, and sternal compression with RV-PA conduit homografts, xenografts, and ePTFE conduits, RVOT reconstruction remains the only option for many patients with CHD's and other valvular diseases (Homann, 2000; Ootaki, 2018; Choi, 2018; Suzuki, 2012; Bonetti, 2019). It is widely understood that current RV-PA interventions are imperfect. According to Kevin Ong et al., “RV-PA conduits continue to play an important role in the surgical repair of CHD but remain plagued by high failure rates, with only half of the patients free from reoperation 10 years after initial conduit implantation” (Ong, 2013), while Mery et al. said, “There were contradicting reports in the literature regarding the durability of the different conduits, and as such, the ideal conduit remains a subject of debate” (Mery, 2016). However, the attributes of an ideal RV-PA conduit include long-term durability and patency, good handling, availability in a wide range of sizes, and low risk of infection (Carreon, 2019).

Many consider cryopreserved pulmonary homografts to be the gold standard for RV-PA conduits. The primary challenge in using these homografts is availability with appropriate sizing, especially in neonates and infants (Christenson, 2004; Reinhartz, 2006; Christenson, 2010). Contributing further to this challenge, it has been shown that non-blood-matched homografts have higher calcification rates, leading to lower durability (Christenson, 2004). Homografts have additional issues with immune response and rejection (Baskett, 1996; Hawkins, 2000). Despite being the gold standard, these conduits carry considerable risk, if they were even available. One retrospective study of the Stanford pediatric surgery group found 25% (22 of 88) of RV-PA implants used alternative conduits due to the lack of homograft availability (Reinhartz, 2006).

Xenograft conduits have become widespread over the past two decades, with the development of many commercial products, including but not limited to the Contegra bovine jugular vein, the Medtronic Freestyle, and the Hancock valve. These products vary widely in their design, with stented and non-stented options, utilizing bovine and porcine tissues and incorporating conduits made from ePTFE, PET, or animal tissue (Corno, 2004; Mery, 2016; Martin, 2018; Alfieris, 2016; Dunne, 2015). Due to availability in a range of sizes, these conduits have become widely utilized, however they have problems with endocarditis, calcification, stenosis, and pulmonary insufficiency (Mery, 2016; Dijck, 2015; Yong, 2015; Peivandi, 2019; Nichay, 2018).

There have been results with bulging sinus ePTFE grafts implanted in pediatric patients; however, the grafts remain challenging to produce and rely on the skill of surgeons to hand sew the device. Ko et al. describe this procedure as “technically challenging and less reproducible” (Yamamoto, 2020; Choi, 2018; Miyazaki, 2007). Beyond these challenges, ePTFE grafts were subject to calcification, neointimal proliferation, and were produced with monocusp, bicuspid, or tricuspid valve configurations depending on the size of the conduit needed and the surgeon completing the procedure (Yamamoto, 2020; Choi, 2018). While the advancement of ePTFE RV-PA conduits represents a positive step forward, there exists a strong need to develop engineered patient-specific devices with excellent hemodynamics that were anti-thrombotic and calcification-resistant.

Only one engineered conduit, the Xeltis Pulmonary Valved Conduit, is currently being developed to address the shortcomings of current RV-PA conduits. Xeltis is a tissue engineering conduit based on the RestoreX™ polymer. The technology may allow complete tissue ingrowth within 12 months to replace the polymer leaflets. While this has been tested in sheep for 12 months and humans for 24 months, it remains to be seen if complete tissue functionality was restored, and the device cannot be evaluated with accelerated wear as it relies on tissue replacement of the RestoreX™ polymer. In the sheep study “tears and fragmentation of the polymer leaflets were observed” at 12 months after the conduit was recovered. The authors had stated conflicts of interest and chose to implant in adult sheep aged 2-4 years despite it being well-understood that implanting in juvenile sheep is the preferred model to test a blood contacting implant against calcification. Calcification is a well-known issue with blood-contacting devices (Bennink, 2018; Flameng, 2006). In all published works no implant used patient-specific anatomy in the conduit design. In the first Xeltis human trial, 11 of 12 patients had moderate to severe pulmonary insufficiency (PI) at 12 months. Leaflet prolapse was cited as the most common cause of PI. The conduit was redesigned in the second trial to prevent PI; however, over the course of 12 months, all six enrolled patients developed some form of PI. As a cohort PI in the second trial was reduced compared to the first trial; however, one patient did have a severe immune response and had to have the device removed and replaced with a Contegra conduit (Prodan, 2021; Morales, 2021). While the initial human trials provide promising results up to 24 months after implantation, the durability of the conduits remains untested and will remain untested until these initial patients age into adolescence and adulthood. Table 2 below highlights the characteristics and shortcomings of these varying versions of RV-PA conduits.

The personalized HA/LLDPE RV-PA valved conduits disclosed herein have immense potential to address the many failings of current options. First, all materials and reagents required to produce the conduit were commercially available and inexpensive. The bio-inspired HA/LLDPE materials were shown to prevent calcification and thrombosis, and a stented HA/LLDPE valve demonstrated no calcification or thrombosis in the pulmonary position of sheep (James, 2015; Heitkemper, 2018; Bui, 2019; Zhang, 2006) after 6 months. The fact that no fixed tissues were used in the device dramatically reduced the potential for endocarditis (Mery, 2016; Christenson, 2010; Perin, 2015). Furthermore, LLDPE has excellent handling characteristics, can be made in any size, and can be easily shaped to fit a specific patient's anatomy. Before the presently disclosed device, no patient-specific pulmonary valved conduits were available. Implanting off the shelf straight conduits often required the surgeon to force the conduit to a curved path increasing concerns for conduit kinking, twisting and compression. In contrast, the device disclosed herein, being a patient-specific curved conduit, considers these areas of compression and made it easier for the surgeon to implant the conduit, thus reducing the stresses in the conduit, making it more durable and improving hemodynamics (Prodan, 2021; Christenson, 2010).

### Discussion of Additional Example 3

Provided herein is a bioinspired HA/LLDPE personalized pulmonary valved conduit that offers a hemodynamically superior and durable solution to the drawbacks of current RVOT reconstruction devices.

The study was conducted to develop personalized biocompatible polymeric right ventricle-to-pulmonary artery (RV-PA) valved conduits for right ventricular outflow tract (RVOT) reconstruction in pediatric patients with congenital heart defects (CHDs). Currently, available valved conduit options include homografts, decellularized allografts, bovine or porcine xenografts, prostheses made from expanded polytetrafluorethylene (ePTFE) and/or polyethylene terephthalate (PET) that were hand sewn intraoperatively, and a newly developed tissue engineering conduit, the Xeltis pulmonary valve (Homann, 2000; Corno, 2004; Ootaki, 2018; Yamamoto, 2020; Prodan, 2021). Given the highly complex and patient-specific nature of the reconstruction, there is a need for durable and customizable RV-PA valved conduits that were personalized for each patient according to pre-procedural computational planning. in the study, linear low-density polyethylene (LLDPE) valved conduit has been developed that can be formed from 3D printed molds and contain a bio-inspired interpenetrating polymer network (IPN) of hyaluronic acid (HA) (James, 2010; James, 2015). HA is a naturally occurring polysaccharide that is highly hydrophilic and is a component of native heart valve leaflets and the endothelial glycocalyx (Prawel, 2014; Reitsma, 2007). Heart valves made from HA/LLDPE were extremely robust: the HA/LLDPE leaflets can survive over 250 million aortic cardiac cycles in-vitro and demonstrate no calcification or thrombosis after 6 months in the pulmonary position of juvenile sheep (Heitkemper, 2018; Bui, 2021). Beyond the excellent mechanical and biocompatible features, LLDPE is widely available, easily shaped, and the process of generating the IPN between LLDPE and HA is a scalable, consistent manufacturing process (James, Hyaluronan (HA) esterification, 2010). The HA/LLDPE IPN materials were utilized herein to develop personalized pediatric valved pulmonary conduits. Bioinspired HA/LLDPE personalized pulmonary valved conduits offered a hemodynamically superior and durable solution to the drawbacks of current RVOT reconstruction devices.
The study established manufacturing protocol for personalized HA/LLDPE pulmonary valved conduits and quantifying kinematics, hemodynamics, and flow field stresses, and performed:
1. Performed manufacturing and assembly of N=10 patient-specific conduits
2. Evaluated Particle Image Velocimetry {PIV}: velocities, viscous and turbulent stresses
3. Evaluated EOA, regurgitant fraction, sinus washout
4. Evaluated Leaflet kinematics and coaptation
5. Evaluated effect of bulging sinus
The study incorporated sewing rings and assessing in-vitro fatigue and calcification resistance.
6. Evaluated Suture pull-out strength
7. Evaluated Material thickness for durability
8. Evaluated ISO Fatigue Testing with hemodynamic {SA 1} reassessment
9. Evaluated In-Vitro Calcification
The study validated in-vivo performance of personalized HA/LLDPE pulmonary valved conduits.
   Evaluated N=6 Pulmonary valved conduit designed for each sheep's anatomy
   Evaluated Doppler transthoracic echocardiography
   Evaluated use of the example device in Angiography
   Evaluated Histology
   Performed Scanning electron microscopy
   Performed Flame atomic absorption spectroscopy

It was observed that the patient-specific HA/LLDPE pulmonary valved conduits offered superior hemodynamics compared to straight commercially available valved conduits. The flows of the patient-customized valved conduits were characterized by different valve configurations in-vitro relative to commercial conduits. Valve design parameters with respect to sinus and leaflet profiles were examined to identify the best patient-specific configuration for n=10 patient models and tested in-vitro per ISO Standards for hemodynamics, including particle image velocimetry.

It was also observed that the patient-specific HA/LLDPE valved conduits endured surgical suturing and met ISO durability standards (ISO 5840-1, 2021) without HA loss or degradation. PET sewing rings were incorporated at each end, and suture pull-out strength was measured. It was determined which conduit thickness profiles resulted in the durability required to meet ISO 5840-1), 2021. Leaflet/conduit fatigue characteristics were examined under in-vitro calcification conditions and accelerated wear testing (AWT) for N=10 patient models. After AWT, conduits were recharacterized for hemodynamics as described above.

HA/LLDPE pulmonary valved conduits exhibited superior durability, anti-calcific, and anti-thrombotic performance in-vivo compared to commercially available conduits. A 12-month juvenile sheep study provided an in-vivo test of the valved pulmonary conduit design and durability. N=6 HA/LLDPE pulmonary valved conduits made specifically for each sheep allowed comparison of durability, calcification, and thrombus to historical controls.

Current commercial RV-PA conduits fall short of meeting clinical needs. The availability of pediatric RV-PA conduits (homografts) is extremely limited (Belli, 2010). Allografts have had disappointing results, with one study showing 40% dysfunction at 5 years (Brown, 2005). Stented and stentless xenograft conduits have failure rates of 35% and 25% at 2 years, respectively (Yuan, 2008). The Contegra xenograft has a “significantly greater risk of late endocarditis” when compared to other valved conduits and also calcifies (Mery, 2016). ePTFE conduits fail due to calcification and require surgeons to intraoperatively hand sew the device resulting in poor quality control (Yamamoto, 2020). The Xeltis pulmonary valve is in early clinical trials and has documented issues with pulmonary insufficiency (Prodan, 2021; Morales, 2021). The highly complex and patient-specific nature of RVOT reconstruction (Capelli, 2018), clinical history of underperforming conduits, and complete lack of standardization demonstrate the need for patient-specific, durable conduits that do not contain fixed tissues and that were made from bio-inspired materials using patient imaging, computational modeling, and good manufacturing practices. No patient-specific pulmonary valved conduits were available today.

### Methods and Materials of Additional Example 3

Discussed below were the HA/LLDPE materials that the conduits were made from, the manufacturing process for the innovative personalized valved pulmonary conduits, and hemodynamic and flow field testing of the novel valved pulmonary conduits.

### In-Vivo Chronic Calcification and Thrombus Study of Additional Example 3

To gain preliminary insight into calcification and thrombus formation in-vivo, the bioinspired HA/LLDPE material was assembled onto surgical valve stents and implanted into the pulmonary position in two young sheep under 1 year old to be evaluated for calcification deposition, thrombus formation, and HA retention. The valve performance was evaluated with a transthoracic echocardiogram (TEE) immediately following the procedure, bi-monthly for the first month, monthly after, and prior to sacrifice. Intracardiac echocardiography (ICE) was also performed prior to sacrifice at 6 months after implantation.

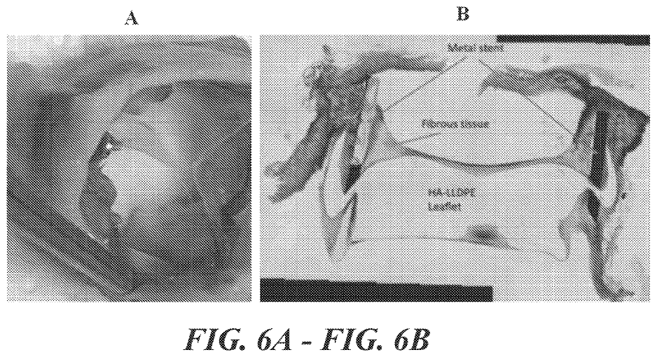

Perivalvar leakage and right ventricle size and function were also evaluated and documented. At no timepoint following the implant procedure were the peak velocities or gradients indicative of stenosis, and no other signs of calcific deposition were present. In both sheep, perivalvular leakage was trivial to none. No abnormalities in ventricular size for function were seen for either animal. Upon sacrifice, the prosthetic valves were explanted and inspected for signs of calcification and other growth (FIG. 6A-FIG. 6B). No calcification was visually present with no thrombus, and the leaflets were clear of thrombus and appeared pristine to the naked eye (FIG. 6A-FIG. 6B), and under the scanning electron microscope (SEM) electron dispersive spectroscopy (EDS) confirmed no calcification was present even at the microscopic level. SEM revealed no signs of damage, tearing, or mechanical failure. TBO staining and FTIR confirmed HA was still present and distributed similarly to unimplanted valves. Some pannus growth was present, but this was a limitation from the bare-metal surgical stent that prevented uniform tissue growth typically seen over trans-catheter stents. The histology showed no signs of calcification or thrombus (FIG. 6A-FIG. 6B).

### Polymeric HA/LLDPE Materials of Additional Example 3

Sodium HA (~700 kDa, Lifecore Biomedical) was complexed with cetyltrimethylammonium (CTA) bromide to create HA-CTA, which was then silylated to create silyl HA-CTA (SHACTA). The hydrophobic SHACTA was introduced into the hydrophobic host (LLDPE) via swelling for 60 min. in a hot (50° C.) SHACTA/xylene solution (1.0% w/v). The LLDPE blown film (Dowlex 2056) was chosen for its high yield, tensile and tear strength, and relatively low modulus and bending strength. The SHACTA introduced into the LLDPE films was vapor crosslinked above 60° C. (2% v/v) 2,4-toluene diisocyanate and xylenes solution. Because the SHACTA was entangled at the molecular level and then crosslinked, the IPN remains upon hydrolysis (which converts the SHACTA back into HA); e.g., the now hydrophilic HA cannot phase separate from the hydrophobic LLDPE.

HA-enhanced LLDPE has been shown to exhibit almost no changes in the tensile properties compared to virgin LLDPE, with some minor exceptions (Bui, 2019). These exceptions were the machine direction HA-treated LLDPE versus transverse direction virgin LLDPE yield stress (7.85±0.27 vs. 7.15±0.09 MPa) and elongation at break of transverse direction HA/LLDPE versus machine direction virgin LLDPE (597±83.5 vs. 445±40.8%). These minor changes indicate the tensile properties were not affected in any substantial way by the treatment or process.

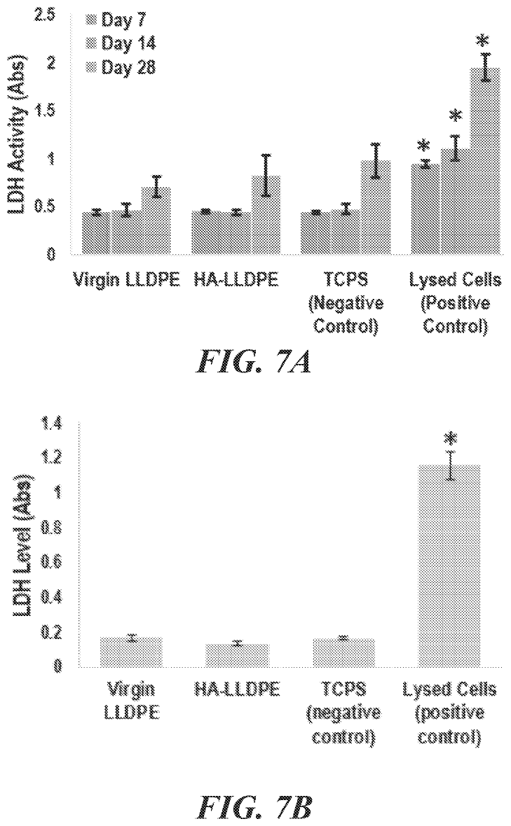

The cytocompatibility of the vapor crosslinked HA/LLDPE was assessed first by growing adipose-derived stem cells (ADSCs) in media incubated with the HA/LLDPE material for 7, 14, and 28 days. The cytotoxic potential was evaluated with a lactate dehydrogenase (LDH) assay. In the second study, HA/LLDPE samples were cocultured with human dermal fibroblasts (HDFs) for 24 hours and evaluated by another LDH assay. LDH is a metabolic enzyme ubiquitous in the cytoplasm of cells. The release of LDH and its concentration in the extracellular environment were correlated to cell death. FIG. 7A-FIG. 7B shows the result of the studies indicating the material is not cytotoxic to either ADSCs or HDFs.

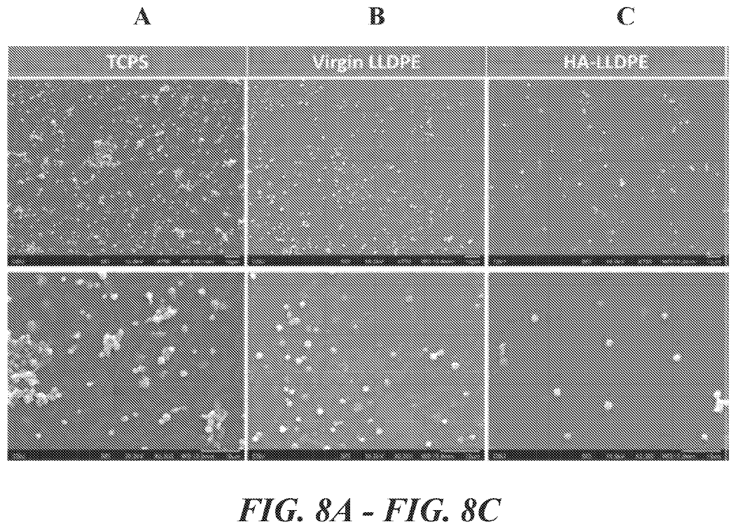

Human platelet adhesion and aggregation were investigated by incubating freshly collected blood plasma for two hours and fixing the adhered platelets to the HA/LLDPE materials using TCPS as a positive control. FIG. 8A-FIG. 8C shows SEM images of the fixed platelets demonstrating a clear reduction in the number of platelets and almost no platelet activation on the HA/LLDPE surfaces compared to the virgin LLDPE and TCPS controls. More extensive explanations of the methods can be found in this reference (Bui, 2019).

### Study Data on Tensile Properties of LLDPE/PET Thermal Bonded at Anastomoses of Additional Example 3

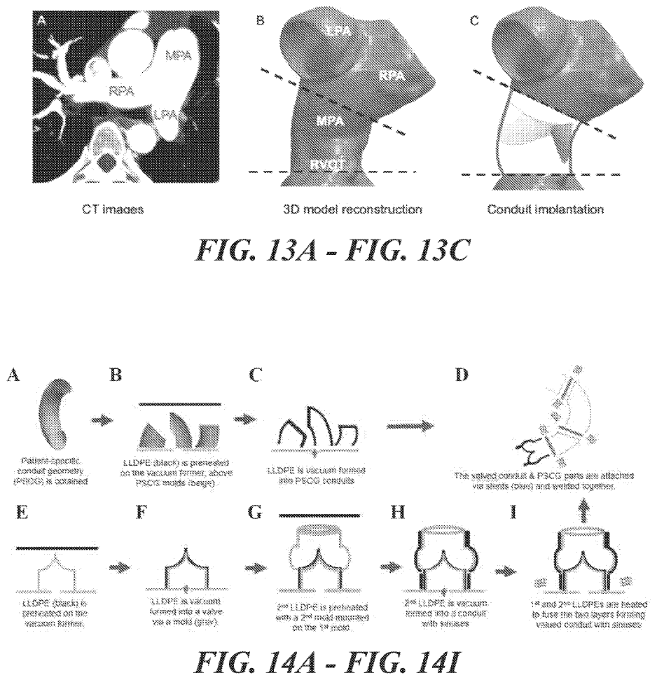

A study tensile test of the composite bond formed by melt pressing PET between LLDPE sheets at a crosshead speed of 25 mm/min. Dog bone punches were taken at the interface between the LLDPE and the PET and in the LLDPE section and pulled to failure. The LLDPE/PET interface (green dog bone) shown in FIG. 13A-FIG. 13C was mechanically similar to the control LLDPE (red dog bone). The interface had a modulus, yield strength, and ultimate tensile strength of 82.63±18.74 MPa, 6.71±1.34 MPa, 8.66±1.36 MPa, and the LLDPE properties were 70.45±7.40 MPa, 6.97±0.99 MPa, 10.65±2.51 MPa. This method of embedding PET in LLDPE will provide a satisfactory process for incorporating a PET sewing into the conduit ends; even though the green interface is slightly weaker, it should withstand the low pulmonary pressures (35 mmHg or 4.7*10⁻³ MPa) (Momenah, 2009). The study used this technique to create PET sewing rings at each end of the conduit that surgeons can use for sewing the anastomoses.

### Assembly of LLDPE Pulmonary Conduit of Additional Example 3

To demonstrate that a polymeric valved conduit can be fabricated using the technology, plain LLDPE was tested rather than the HA/LLDPE IPN because the HA is incorporated into the LLDPE after forming, as shown below. FIG. 10A-FIG. 10J shows the results of the thermoforming process steps to assemble the LLDPE valved conduit. LLDPE (80 μm thick) sheet was mounted on a vacuum former, preheated for 30 seconds, and vacuumed formed into a valve using a 3D printed mold. The process was repeated with another LLDPE sheet thermoformed around a tubular mold that was mounted on the valve, which created the conduit. Further heating of the valved conduit along its wall fused the two LLDPE layers together. FIGS. 10 (a, b, & e) shows pictures of the clear plastic LLDPE valved conduit.

### Advanced Manufacturing Method for Shaping Leaflets and Making Curved Conduits of Additional Example 3

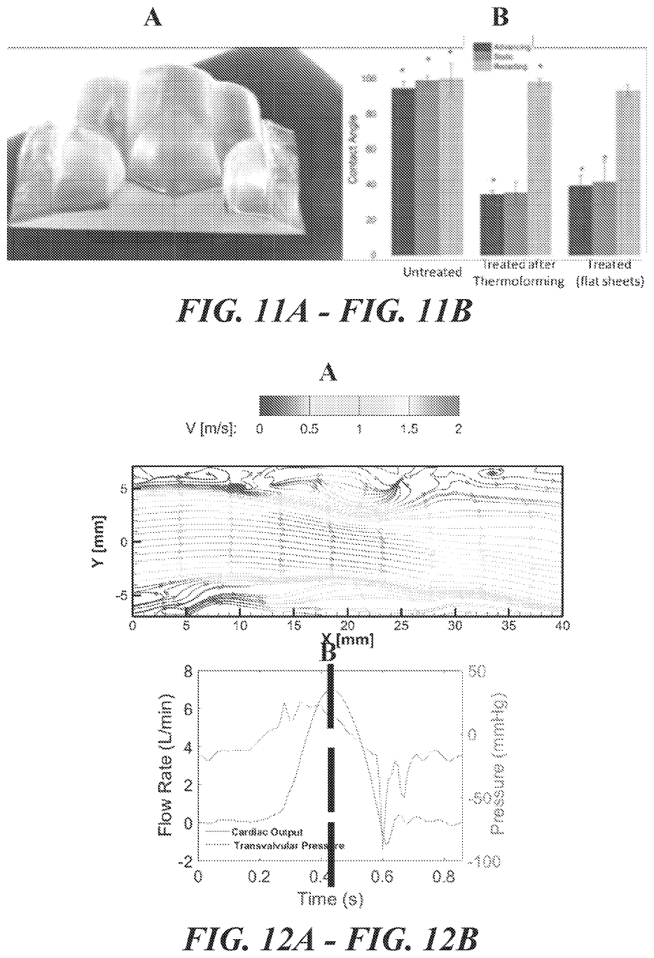

The 3D-shaped leaflets will help with performance and durability based on FEA simulations. Thermoforming, a vacuum-forming process, can be used to shape the LLDPE (FIG. 10A-FIG. 10J) before it is treated with HA. The form was 3D printed with a dental grade resin (Orthotough M, EnvisionTEC) to achieve feature resolution and a smooth surface finish. The vacuum-forming process heats the LLDPE up to approximately 100° C. for one minute before shaping over the mold or tool. Treating the flat LLDPE with HA and then thermoforming the leaflet shape, HA is lost from the surface of the material, and the contact angle increases considerably. If the LLDPE is thermoformed first and then treated with HA as described above, the leaflet maintained its 3D shape and fits the original mold, containing HA (based on FTIR) and contact angle (FIG. 11A-FIG. 11B).

Neither the heat shaping nor welding the thermoformed shape into a cylinder for HAPTAV assembly significantly changed the LLDPE crystallinity (measured by differential scanning calorimetry, DSC) or tensile properties (ASTM D 882-18).

### In-Vitro Hemodynamic Testing of LLDPE Pulmonary Conduit of Additional Example 3

Three LLDPE valved conduits of different geometries were fabricated and analyzed to demonstrate the ability to make the valved conduit in different sizes and shapes. The samples were implanted in an in-vitro model (FIG. 27) and tested under pulsatile pulmonary flow conditions (8/25 mm Hg diastolic/peak systolic pressure). A working fluid of 60/40 water to glycerin (99% pure glycerin) was used to provide density and kinematic viscosity comparable to blood at 1060 kg/m³ and 3.5.10⁻⁶ m²/s, respectively. The in-vitro model also required the conduit samples to be submerged in the blood analog (60% glycerin), similar to the right heart condition. Pressure gradient, as well as flow waveform, were collected. From these data, the mean transvalvular pressure gradient (11P), peak transvalvular pressure gradient (peak 11P), effective orifice area (EOA), and a regurgitant fraction (RF) were computed for each of the valved conduits. FIG. 5) (c, d, h, & i) shows representative images of the pulmonary valve fully closed and opened while inside the pulsatile flow setup. Table 1 shows the hemodynamic data for the LLDPE valved conduits. Overall, the shorter adult (26 mm) conduit performed better, as shown by the lower 11P, higher EOA, and lower RF. In both adult conduits, the regurgitation is mild (≤20%). The pressure gradients of the LLDPE conduits were comparable to that of Contegra bovine jugular conduits, which have a mean trans-conduit gradient of 9.6±5.3 mm Hg during hospital discharge and 13±8 mm Hg after a mean of 85-month follow-up (Sfyridis, 2011). The acceptable peak transvalvular gradient of the Contegra conduit was observed to be 18±9 mm Hg within a one-year follow-up. The pediatric conduit (14 mm) has a comparable 11P to the shorter adult conduit (13.6 mm Hg), with a similar peak 11P to the longer adult conduit. The EOA for all samples was close to the minimum device performance required value for the appropriate valve size (between 1.45 and 1.70 cm² for 26 mm ID and approximately 0.47 cm² for 14 mm ID as extrapolated from the ISO 5840). The EOA is the estimated area of the jet at the vena contracta, and it is inversely correlated to the pressure drop across the valve. Therefore, a higher EOA corresponds to a more efficient and desirable valve. The low cost and abundant supply of LLDPE make the device an attractive alternative to currently available bovine and porcine fixed tissue conduits.

A more in-depth analysis of the flow study of the pediatric valved conduit sample (14 mm) from table 1 is shown in FIG. 12A-FIG. 12B. The cardiac output and transvalvular pressure (pulmonary/ventricular) for one cardiac cycle are shown on the left, while particle image velocimetry (PIV) data during peak systole is shown on the right. PIV image was captured from 14 repeated phase-locked measurements of the cardiac cycles, and the flow was seeded with florescent PMMA-Rhodamine B particles (diameter 1-20 μm). The PIV velocity profile shows peak jet velocity is 2 m/s, which is close to the mean velocity at follow-up (1 to 31 months) for the Contegra Bovine Jugular RV-PA conduit (1.91±0.31 m/s) (Purohit, 2004).

TABLE 3
Hemodynamic data and in-vitro setting of the LLDPE pulmonary valved conduit. 26 mm and
14 mm valved conduits were tested under adult and pediatric conditions, respectively.

| | | Cardiac | Heart | Systolic | flP | | Peak lP | | | | (RF) | |
|---|---|---|---|---|---|---|---|---|---|---|---|---|
| Diameter | Length | Output | Rate | Phase | (mmHg) | | (mmHg) | | (EOA) | | (%) | |
| (mm) | (mm) | (L min) | (bpm) | (%) | mean | std | mean | std | mean | std | mean | std |
| 26 | 35 | 5 | 60 | 33 | 12.9 | ±0.31 | 17.4 | ±0.39 | 1.59 | ±0.02 | 8.67 | ±0.86 |
| 26 | 45 | 5 | 60 | 33 | 18.1 | ±0.25 | 24.7 | ±0.28 | 1.33 | ±0.02 | 9.53 | ±0.49 |
| 14 | 65 | 1.5 | 70 | 45 | 13.6 | ±0.72 | 24.9 | ±1.17 | 0.46 | ±0.13 | 2.83 | ±0.74 |

TABLE 4
Comparing Current PV-RT Conduit Technologies to Exemplary
Personalized HA/LLDPE Valved Pulmonary Conduit

| | | | | | | Success |
|---|---|---|---|---|---|---|
| Conduit | Exemplary | Existing Technologies/ | | | | Criteria |
| Characteristics | Technology | Historical Controls | | | | Outcomes |
| (citations) | HA/LLDPE | Homograft | Xenograft | ePTFE | Xeltis | (p < 0.5) |
| Free from Intro- | Yes | Yes | Yes | No | Yes | Achieved in |
| Operative | | | | | | study data |
| Constructive | | | | | | |
| Personalized to | Yes | No | No | No | No | SAs 1 and 2 |
| Patient Anatomy | | | | | | |
| Calcification- | Yes | No | No | No | ? | Less in- |
| Resistant | | | | | | vitro and |
| | | | | | | in-vivo (SA |
| | | | | | | 3) |
| | | | | | | calcification |
| | | | | | | than |
| | | | | | | controls |
| Long Term | Yes | No | No | No | Concerns | Superior to |
| Patency | | | | | | historical |
| | | | | | | controls |
| | | | | | | (SAs 2 and |
| | | | | | | 3) |
| Long Term Valve | Yes | No | No | Yes | No | Superior to |
| Function | | | | | | historical |
| | | | | | | controls |
| | | | | | | (SAs 2 and |
| | | | | | | 3) |
| Free from | Yes | No | No | Yes | Concerns | Less than |
| Immunological | | | | | | historical |
| Response | | | | | | controls |
| | | | | | | (SA 3) |
| Free from | Yes | No | No | No | Yes (24 | Less |
| Endocarditis/Low | | | | | mo.) | endocarditis |
| Risk of Infection | | | | | | than |
| | | | | | | historical |
| | | | | | | controls |
| | | | | | | (SA 3) |
| Anti-Thrombotic | Yes | No | Yes | Yes | Yes (24 | Less in- |
| | | | | | mo.) | vivo |
| | | | | | | thrombus |
| | | | | | | than |
| | | | | | | historical |
| | | | | | | controls |
| | | | | | | (SA 3) |
| Excellent | Yes | Yes | Yes | Yes | Yes | Easy to |
| Handling | | | | | | place and |
| Characteristics | | | | | | suture |
| Abundant Supply | Yes | No | Yes | ? | ? | Achieved in |
| | | | | | | study data |
| Shaped Leaflets | Yes | Yes | Yes | No | Yes | Achieved in |
| | | | | | | study data |

### Study Phase 1 of Additional Example 3: Establish Manufacturing Protocol for Personalized HA/LLDPE Pulmonary Valved Conduits and Quantify Kinematics, Hemodynamics, and Flow Field Stresses.

### Patient-Specific HA/LLDPE Pulmonary Valved Conduits Offered Superior Hemodynamics Compared to Straight Commercially Available Valved Conduits.

The study characterized the flow and structural response of the novel patient-customized valved conduits with different valve configurations in-vitro relative to commercial conduits with similar valve annulus diameters. Valve design parameters with respect to sinus and leaflet profiles were examined to identify the best patient-specific configuration for n=10 patient models and tested in-vitro as per ISO 5840, 2021 for hemodynamics, including particle image velocimetry.

As the study data has shown that HA enhancement does not change the mechanical properties of LLDPE and that thermoformed LLDPE can be treated with HA without losing its shape, plain LLDPE, (2045G, 80-μm thick) was used in Phase 1. In this Phase, the term LLDPE valved conduits refer to the ten patient-specific conduits that would be created from patient computed tomography (CT) scan data and the plain LLDPE control. The main goal of this Phase is to improve the hemodynamic performance of the customized LLDPE valved conduits by optimizing conduit geometry. The study is an iterative process that involves (1) improving the LLDPE valved conduits by adjusting leaflet geometry and testing the presence of a sinus; (2) conducting comprehensive hemodynamics testing and time-resolved particle image velocimetry (TRPIV). All results were compared to that of straight LLDPE conduits and published literature on commercially available valves such as the Contegra and Hancock.

Personalized HA/LLDPE RV-PA Conduits were Created from Patient CT Data.

The patient-specific valved conduit design primarily focuses on understanding the optimum conduit valve annulus diameter and curvatures to maximize the functionality of the conduit for the patient's heart anatomy and improve the case with which clinicians can surgically implant the conduit. Using pre-surgical CT images, a 3D model of the patient's right ventricle/right ventricular outflow tract is shown in FIG. 13A-FIG. 13C. Approved protocols were performed that collected deidentified imaging datasets of children with complex congenital heart defects. That existing database was at the disposal of the study. The patient-specific pulmonary trunk was segmented and constructed using Mimics Research 18.0 (Materialise, Leuven, Belgium). The dimensions of the different anatomical structures obtained from the segmented model were used to serve as the starting point for deciding the conduit shape, length, and valve annulus size. Using the 3D RVOT model, the conduit implantation can be visualized, and design changes can be made to reduce high shear stress areas at sharp curvatures. Thus, a patient-specific LLDPE valved conduit can be designed from pre-operative CT scans. In order to manufacture the customized conduits, a multi-step thermoforming process demonstrated in FIG. 14A-FIG. 14I was used. Part of the process (see steps 1b to 5b) was used to create the valved conduits in the study data. Steps 1a to 3a (of FIG. 14I) were tested along with welding stents of the patient-specific conduit. The study tested these methods based on the previous work where a metal alloy stent was incorporated onto the HA-enhanced polymeric transcatheter heart valves (Bui, 2021; Bui, Design and Fabrication, 2021). In FIG. 14A-FIG. 14I, the end of the stents (blue lines) were not located at the valve but between the modular sections of the conduit body. The stents were placed between the LLDPE at the modular junctions, and the LLDPE welded together, as seen in FIG. 14A-FIG. 14I. This would embed the stent, reinforcing the conduit body in the areas of the highest curvature.

### LLDPE Pulmonary Valved Conduit Assembly: Parameters—Sinus and Leaflet Geometry:

The assembly model described and presented in the study (FIG. 10A-FIG. 10J) was utilized but with varying designs to make the patient-specific LLDPE valved conduits. The conduits were tested with and without a sinus to analyze the hemodynamic effects. Incorporation of a bulging sinus can reduce pressure gradients, improve EOA, and lower peak velocities. Furthermore, a bulging sinus may generate a vortex flow similar to that created by the sinus of Valsalva supporting valve closing (Miyazaki, 2007; Sadri, 2021). In order to incorporate a sinus geometry into the valve, the molds used to create the valved conduits were altered. A small profile for leaflets can lead to increased regurgitation. In this phase, the study manufactured and tested the customized conduits with aspect ratios of 0.5, 0.65, and 0.8. These were optimal values based on the literature (Thubrikar, 1990; Sluysmans, 2005). This parameter will also help optimize the leaflet geometry to avoid “pin-wheeling” that is known to induce additional structural stresses within the leaflets, impacting long-term durability. The leaflet perimeter shape was studied by comparing closing dynamics and regurgitation levels for flat-edged leaflets to circular-edged leaflets. This will guide improvement in leaflet coaptation and reduce regurgitation. The axial length of the leaflet at the tip was adjusted to be higher than the length on the commissures. The study evaluated 3 levels of differences: 0 mm, 2 mm, and 4 mm, respectively. The higher the difference, the more leaflet area is available for coaptation at the center.

There is a close correlation between body surface area (BSA) and the sinus of Valsalva, which is 26.79±6.59 mm/m² (r−0.886, p<0.001) when BSA is between 0.5 and 1.0 m² (ref⁶⁴). Interestingly, all heart valve diameters were shown to be linearly related to the square root of BSA⁵⁶. The relationship between the main pulmonary artery (MPA) (cm) and BSA (m²) is shown in the following equation (r−0.908):

$$M P A = ( 1.653 ) ( B S A0 \text{.5} ) + 0.033 M P A = 1.653 B S A0 \text{.5} + 0.033$$

Based on the above relationship, the sinus diameter for each of the designs was calculated and incorporated into their respective designs. It is noted that the sinus size calculation is based on the sinus of Valsalva located at the aortic position. Therefore, the study studied three different sinus sizes for each of the conduits. For example, a 14 mm valve annulus was studied with 18.3, 17.3, and 16.3 mm sinus sized (Hatoum, 2020). Sinus size may affect fluid motion and sinus washout, which can contribute to flow stasis and thrombosis. By decreasing sinus width by approximately 31%, the peak fluid velocity at the sinus has been shown to quintuple⁶⁶. It is important to study the influence of sinus geometry on the hemodynamic performance of the conduit.

### Hemodynamics and Kinematics Characterization.

Hemodynamics and kinematics of the different configurations of the personalized valved conduits (defined in Task 1.1) were compared to that of straight conduits and a clinical Contegra or Hancock of similar size (Christenson, 2010; Morray, 2017). These measurements were performed using the dynamic in-vitro right heart simulator system (FIG. 27), which contains a transparent anatomical chamber to place conduit samples. This anatomical model, personalized for each patient, will also have similar geometry to the specific patient's native RV-PA for highly controlled comparison while permitting full optical access (conduits were transparent). Viscosity and refractive-index matched water-glycerin-NaI Blood analog (Leo, 2006; Dasi, 2008) were used as the flow loop fluid. The flow loop for each conduit was modified by tuning it to patient-specific physiological conditions according to Table 3 below. For each condition, the flow field downstream was measured using TRPIV in addition to bulk hemodynamic performance parameters (EOA, pressure gradient, and regurgitant fraction) and high-speed videos of marked leaflets.

### Experimental Methods of Additional Example 3

### Valve Kinematics Measurements

Conduit leaflet motion was mapped in detail using high-speed video (LaVision Inc.). Leaflet opening and closing times were compared between LLDPE valved conduits and data from numerous other clinical prosthetic valves. Using a marking dye (Thermoelectron Corporation), a regular array of markers were placed on the leaflet surface. These markers were tracked over the cardiac cycle for leaflet kinematics and stretch computations. Two views were mapped into the single high-speed camera using mirror arrangements to gain a stereoscopic view of each leaflet. This image acquisition was gated to the acquisition of hemodynamic data through the pulse programmer. At the end of dynamic image acquisition, without draining fluid from the loop, both the ventricular and pulmonary chambers were exposed to atmospheric pressure, and the valve was allowed to assume its static, zero-transvalvular pressure configuration. Images of the valve leaflets in this state were captured, and the corresponding leaflet geometry was used as the zero-pressure reference configuration for stretch computation. The arrays of markers at the region of interest were tracked using a custom MATLAB program from 2D images from both cameras. These 2D coordinates of the markers were converted to 3D coordinates by Direct Linear Transformation (Hartley, 2003) through the resolution of the relative angle between the two views. To calibrate for this angle between the stereoscopic-views, a 5 mm metal cube was inserted into the chamber at the location of the leaflets, and images of the cube were captured from both views. Coordinates of the seven visible vertices of the cube will then be used to compute the view angle and position. Shell-based 2D iso-parametric finite element shape functions were used to fit leaflet surface geometry described by the 3D coordinates of markers. These shape functions can then be used to compute the dynamic principal stretches. The unstretched reference state was taken as the state when the flow loop was stopped, and pressure in both the ventricular and atrial chambers was equilibrated. The study used Smith's methodology for the computation of stretch and strains (Smith, 2000).

### Valve Hemodynamic Performance:

All standard prosthetic valve hemodynamic measures (Yoganathan, 2004) such as effective orifice area (EOA), regurgitant volume fractions, mean and peak pressure gradient, valve opening and closing times will define the bulk hemodynamic performance endpoints for the above conditions (ISO 5440-1, 2021). These were evaluated on each of the valved conduits tested, and they were implanted in an in-vitro model (FIG. 27). To accurately capture the physiology of the patient, each conduit was tested in the pulsatile flow system under corresponding conditions as shown in table 3 (Yoganathan, 2013). The pulmonary chamber for the in-vitro model was designed according to the patient's RVOT geometry to simulate realistic flow conditions. A working fluid of 60/40 water to glycerin (99% pure glycerin) was used to provide density and kinematic viscosity comparable to blood at 1060 kg/m³ and 3.5×·10⁻⁶ m²/s, respectively. Pressure gradient, as well as flow waveform, were collected at a sampling frequency of 100 Hz for sixty consecutive cardiac cycles.

TABLE 5
Different pulsatile flow test conditions for analyzing
the hemodynamics of the LLDPE valved conduits.

| Population | Systolic | | | |
|---|---|---|---|---|
| group for | duration | Pulmonary artery | Heart rate | Cardiac output |
| patients | (%) | pressure (mmHg) | (beat/min) | (L/min) |
| Newborn | 50 | 20 | 150 | 1 |
| Infant | 50 | 20 | 120 | 2 |
| Toddler | 45 | 20 | 100 | 3 |
| Child | 40 | 20 | 80 | 3.5 |
| Adolescent | 35 | 20 | 70 | 5 |
| Adult | 30 | 20 | 60 | 5 |

### Flow Field Measurements:

Detailed measurements of the turbulent velocity field were acquired in the immediate vicinity of the valves (both upstream and downstream). Briefly, TRPIV methods include the use of the PIV system (LaVision, Inc) for data acquisition and processing. The flow loop fluid was seeded with 1-20 microns of melamine resin particles coated with Rhodamine-B. The Nd:YLF Single Cavity Diode Pumped Solid State High Repetition Rate Laser (Photonics Industries) was used with a combination of lenses to illuminate a 0.2 mm thick measurement plane through the conduit holder. A double frame CMOS camera (Photronix, Inc) was positioned orthogonally to the laser sheet to gain a good field of view of the particle-laden flow distal to the leaflets. To correct image distortion due to camera angle and chamber geometry, a calibration grid was inserted into the field of view region, and DaVis (LaVision, Inc) image calibration algorithm was applied to images of the grid. Measurements were acquired across a stack of PIV slices spanning the valved conduit with slice spacing of 3 mm. For each slice, an ensemble of approximately phase-locked 500 measurements was captured at a given cardiac phase to enable statistical characterization of the flow field and capture cycle-to-cycle variations in the flow. Simultaneous ventricular and pulmonary pressure measurements were made for at least five hundred phases of the cardiac cycle. The results will yield viscous and turbulent shear stress estimates in the vicinity of the valve. Details of the experimental setup, data acquisition, and processing were described elsewhere (Leo, 2006; Dasi, 2007; Dasi, 2008; Ge, 2008). The PIV measurements were gated with the pulse programmer of the flow loop and programmed to record five hundred phases over the cardiac cycle. Detailed characterization of the data, which includes viscous as well as turbulent stresses, was performed with the protocols and algorithms published by M-PI Dasi for heart valves (Leo, 2006; Dasi, 2008; Ge, 2008).

### Phase 1 Discussion

The personalized conduits were manufactured and treated with HA successfully for use in Phases 2 and 3, based on the study data. Given the good bulk hemodynamic performance shown in study data and literature (Heitkemper, 2019), low or equivalent levels of turbulent and viscous shear stresses were expected from the patient-specific LLDPE valved conduits while demonstrating superior hemodynamics.

### Additional Consideration for Phase 1

The patient-specific conduits were thermoformed into different parts, welded together, and structurally supported via a stent frame as described in FIG. 14A-FIG. 14I. If the stented portions do not provide enough support to ensure a functional conduit or adversely affect the performance (e.g., increasing turbulence and gradient), the study experiment with various reinforced rings as demonstrated in the literature and commercially available RV-PA conduits (Schreiber, 2009; Bentham, 2015). For example, the Hancock bioprosthetic valved conduits contain an annulus ring to preserve the orifice shape.

Five hundred phase-locked images have been shown to be adequate for turbulence characteristics (Leo, 2006; Dasi, 2008; Ge, 2008). However, if potential statistical convergence poses a problem, the study increased the total number of phase-locked images to 1000 at the expense of reducing the number of cardiac phases; (2) Stereo-photogrammetry—If the markers on the leaflets were not visible due to limited exposure times associated with high speed video capture, fluorescent markers were used to improve signal-to-noise characteristics; (3) It is anticipated that the experimental results for the three leaflets not to be symmetric due to naturally excited asymmetric flow instabilities. Any asymmetries in the averaged turbulence quantities were correlated to asymmetries in the HV geometry. HV geometry was assessed using standard imaging. Since the leaflet motion may also respond to turbulent fluctuations in pressure and velocity, alternative approaches were defined, such as quantifying the mean positions from kinematics data over multiple heart beats.

### Phase 2 of Additional Example 3: Incorporate Sewing Rings and Assess In-Vitro Fatigue and Calcification Resistance.

Patient-Specific HA/LLDPE Valved Conduits Endured Surgical Suturing and Meet Valve Durability Standards (ISO 5840-1) without HA Loss or Degradation.

PET sewing rings were incorporated at each end, and suture pull out strength was measured. The study determined conduit thickness profiles result in the durability required to meet (ISO 5840-1). The study examined leaflet/conduit fatigue characteristics under in-vitro calcification conditions and accelerated wear testing (AWT) for n=10 patient models. AWT conduits were recharacterized for hemodynamics as described in Phase 1.

Incorporate PET Sewing Rings into RV-PA Conduit

PET (a.k.a. Dacron®) is known to have excellent handling characteristics and is easily sutured without leakage leading to its use as a sewing ring in many cardiovascular applications, including RV-PA conduits. Therefore, the study incorporated PET sewing cuffs at conduit openings in the following manner. (1) LLDPE formed at the anastomosis is preheated on the PSCG (patient-specific conduit geometry) mold. (2) A PET sewing ring is slipped around the LLDPE cylinder at the anastomosis. (3) A second sheet of LLDPE is thermoformed around the initial LLDPE geometry embedding the ring between the LLDPE sheets. (FIG. 11). PET has a melting point of 260° C. and is insoluble in xylenes. Thus, it tolerates LLDPE bonding and the HA treatment process.

### Suture Study

To determine that the bonded LLDPE/PET can endure surgical suturing, a suture pull out test was performed. Briefly, an example thermoformed LLDPE/PET conduits is fabricated to have a single 4-0 prolene suture placed at the edge of the conduit at the location of the PET ring. A tensile test performed at a crosshead speed of 1 mm/second was performed while clamping the suture and the opposite side of the ring being tested. The amount of force needed to remove the suture was recorded and evaluated using the normalized cross-sectional area to determine stress. The study compare pullout force to historic data of suture pullout from cardiovascular tissue (Paul, 2017).

### Fatigue Measurements and Calcification

The ten patient-specific valved conduits treated with HA, as described in the study data, were placed in a heart valve accelerated wear tester (AWT) (BDC Laboratories' VDT-3600i) and subjected to physiological pulmonic load at a pulse rate of 15 Hz to cycle levels of 50×10⁶, 100×10⁶ cycles, 200×10⁶ cycles. Briefly, the AWT is specifically designed to open and close the valve conduit at an accelerated rate by circulating water and a calcification solution (CaCl₂ 2.1 mM, Ca Total 2.1 mM, KH₂PO₄ 1.0 mM, PTotal 1.0 mM, NaCl 115 mM, and KCl 4.0 mM) via a fluid actuator coupled with a pressure and flow condition system. The loading pressure and waveform were modulated by the tuning module in the system. The upstream and downstream pressures were set to ensure a consistent peak differential pressure across the closed valve (≥25 mmHg), which was monitored continually throughout the study.

A custom-made fixture was fabricated to properly mount the valved conduit into the AWT. After each fatigue test, bulk hemodynamic properties described above were re-evaluated under the conditions detailed above in Phase 1. Structural damage was assessed macroscopically and microscopically. En face high-speed imaging data at every fifty million cycles, and both pre-testing and post-testing data containing microscopic inspection and photographic documentation of the test samples were collected. Calcification of the samples were quantified via flame absorption spectrometry and imaged using SEM (Kiesendahl, 2020; Boloori, 2014). HA treatment of the conduit was evaluated using FTIR (Nicolet 6700) and toluidine blue-O staining (Bui, 2019).

If early failure (before 200 million cycles) of the conduit occurs, the study built conduits from thicker LLDPE in the areas of failure. 80-micron thick LLDPE was used, and there were run valves made from this material to 250 million cycles. Conduits in the study were made from 80-micron thick LLDPE but valves were made from 120-micron and 160-micron thickness LLDPE. Due to the modular nature of the conduit construction process (FIG. 14A-FIG. 14I), any failure point can be made with thicker material without altering other portions of the conduit.

### Phase 2 Discussion

Suture pullout force was greater than historic cardiovascular tissue data. Conduits were more durable and showed no calcification compared to commercial controls and the straight conduits. There was no significant LLDPE damage or HA loss during the AWT over two hundred million cycles.

### Potential Problems and Alternative Approaches for Phase 2

If the suture pullout force is less than that of native tissue, thicker LLDPE and alternate weaves of PET were assessed. HA degradation or loss was not expected; however, if HA loss occurred the study increased the crosslinking density of the IPN by increasing the concentration of the toluene diisocyanate in the vapor crosslinking process. If significant valve damage occurs at a cycle rate of 15 Hz, studies were repeated at lower cycles (e.g., 10 Hz). Given the viscoelasticity of the LLDPE material, high-frequency testing may limit stress relaxation, subjecting the valve to stresses significantly greater than in-vivo.

### Phase 3 of Additional Example 3: Validate In-Vivo Performance of Personalized HA/LLDPE Pulmonary Valved Conduits.

### HA/LLDPE Pulmonary Valved Conduits Exhibit Superior Durability, Anti-Calcific and Anti-Thrombotic Performance In-Vivo Compared to Commercially Available Conduits.

In this Phase, the study conducted a 12-month juvenile sheep study to provide an in-vivo test of the valved pulmonary conduit design and durability. N=6 HA/LLDPE pulmonary valved conduits made specifically for the sheep will allow comparison of durability, calcification, and thrombus. Historical controls were used from published data on other RV-PA conduits in sheep, to reduce live animal use and reduce costs. Juvenile sheep were a standard animal model of choice to assess RV-PA conduits for calcification, neointima formation, and hemodynamic and angiographic assessment (Herijgers, 2002; Azakie, 2020).

A single valved conduit, personalized for each sheep based on CT data, was implanted in the pulmonary outflow tract of six sheep for 12 months. This conduit was fabricated based on the best design outcomes from Goals 1 and 2. The native leaflets were excised from the sheep to enable the HA/LLDPE conduit to operate as a total replacement RV-PA conduit.

### Anesthetic Procedures

The sheep was induced for anesthesia by Valium, Midazolam, or propofol along with ketamine through a venous catheter placed in an ear vein. Following induction, the sheep was intubated and transferred to Isoflurane in an oxygen inhalant titrated to maintain a plane of surgical anesthesia. See Table 1 in the vertebrate animal section.

### Surgical Technique

Sheep was placed in right lateral recumbency after induction of general anesthesia. A left intercostal thoracotomy was performed in the third intercostal space. The descending aorta was exposed and prepared with two mattress sutures for the placement of an aortic cannula. The pericardium can then be opened over the right ventricular outflow track. Heparin was administered intravenously at the dose of 300 UI/kg. A two-stage venous return cannula was placed from the apex of the right atrial appendage into the caudal vena cava and the right atrium. The sheep can then be placed on a bypass with a beating heart. The pulmonary artery can then be exposed and incised 5 mm distal to the pulmonary valve. After removal of the pulmonary valve the valved conduit was sutured in place with two end-to-end anastomosis with 4-0 prolene sutures. After implantation of the valve, the sheep was weaned off bypass and the cannula removed.

### Follow Up

The sheep can undergo transthoracic echocardiographic (TTE) evaluation after stabilization to assess valvular and right ventricular function. Indices of valvular performance can include transvalvular flow velocity and pressure gradient (stenosis), color-flow and spectral Doppler analysis for valve regurgitation, M-mode analysis of leaflet motion, and 2-D analysis for the presence of thrombus. Leaflet function and the presence of thrombus were evaluated by echocardiography at the time of implantation, as well as at 1 month, 3 months, 6 months, and 12 months (prior to sacrifice). Upon explanation, leaflets/conduit were photographed for measurements of thrombus free surface, and the dimensions of the leaflet were compared with pre-implant dimensions. Four sections of the conduit were taken as follows: one section at the proximal anastomosis, parallel to blood flow, to view the transition from tissue to the PET sewing ring and the transition from the sewing ring to the HA/LLDPE conduit, one section at the distal anastomosis parallel to blood flow to view the same transition as above, one section through the valve leaflets parallel to blood flow, and one section above and below the leaflets perpendicular to blood flow. Hematoxylin and Eosin stain were used to assess neointimal formation and thrombosis, while a Von Kossa stain was used to assess calcification. The sections not used for histology can undergo either SEM analysis to visualize clot formation or can undergo flame atomic absorption spectroscopy to quantitively assess calcification. Furthermore, sections of the conduits were examined for HA content using thermal gravimetric analysis and toluidine blue-O staining.

### Phase 3 Discussion

The personalized HA/LLDPE conduits in the study showed little or no thrombus and little to no calcification, demonstrating superior performance compared to all currently available RV-PA conduits. There were no signs of mechanical degradation or fatigue damage in the HA/LLDPE conduits.

### Phase 3 Consideration

Although valve sizing is a potential problem for testing any prosthetic conduit in a live animal, this problem did not arise because the study personalized each conduit to each sheep's anatomy. If thrombus is noted at the end of week 1 the study deployed anti-platelet therapy, failing which the study used a low-dose anti-coagulation therapy. Once the appropriate anti-coagulation therapy is determined, the sheep study can continue.

### Additional Example 4: Expandable Conduit Device

Provided herein is an expandable conduit device. Specifically, the conduit device of Example 4 is an expandable pulmonary conduit that can accommodate somatic growth in pediatric patients who require right ventricular outflow tract (RVOT) reconstruction. In some examples, the expandable conduit device may include any of the features or qualities described in any of the previously described conduit device examples.

### Example Materials

The valved conduit device comprises a conduit comprising HA-LLDPE with varying thicknesses. It also comprised a valve having leaflets comprising HA-LLDPE and a stent/frame comprising a metallic and polymeric material, wherein the metallic materials included nitinol, Co—Cr, stainless steel, or any combination thereof. The device also comprised sewing rings made of materials that include but are not limited to medical grade fabric (Dacron-PET), elastic rubber-like material, or polymeric material (e.g., polycaprolactone).

### Device Features

Generally, device features include expandability—an increase in length and diameter to account for somatic growth in pediatric patients. The expansion was achieved using a transcatheter balloon; durability—all materials used were durable and met ISO standards for durability; hemodynamic expandability—excellent hemodynamic performance at all growth stages; flexibility—the ability to be curved to account for complex patient-specific congenital heart disease anatomies; and low cost—low cost and reproducible/reliable method of manufacturing and low cost to make it accessible and affordable globally.

An example expandable conduit device 1900 is illustrated in FIG. 19 and includes an expandable thermoformed conduit 1902 formed of a first low melting point thermoplastic material suitable for thermoforming. The thermoformed conduit includes a first end 1904 and a second end 1906 and defines a channel 1908 therebetween. The expandable conduit device 1900 is configured to be surgically implanted in a subject's heart or blood vessel, wherein the channel 1908 is configured to permit ingress and egress of blood.

As shown in FIG. 20, the expandable thermoformed conduit 1902 is configured to have a first configuration 2010 having a first circumferential profile C1 and a second configuration 2020 having a second circumferential profile C2. The second circumferential profile C2 has a larger cross-sectional area than the first circumferential profile C1. In some uses, the expandable thermoformed conduit 1902 may be expanded from the first configuration 2010 to the second configuration 2020 during a single surgical procedure. In other uses, the expandable thermoformed conduit 1902 may be expanded from the first configuration 2010 to the second configuration 2020 over the course of two or more surgical procedures, passing through various intermediate circumferential profiles therebetween.

In some examples, the expandable thermoformed conduit 1902 is configured to expand to the second circumferential profile C2 via plastic deformation when mechanically induced by an expandable device. As used herein, mechanically inducing includes positioning an expandable member in a position within the channel of the expandable thermoformed conduit 1902 and expanding the expandable member so that it contacts an inner wall of the channel and exerts an outwardly directed force thereupon so as to adjust the cross-sectional area of the expandable thermoformed conduit 1902 at the first position from the first circumferential profile C1 to the second circumferential profile C2. Example expandable devices may include balloons, stents, or catheters. Other methods may also be utilized to assist in expanding the expandable thermoformed conduit 1902, such as the application of heat.

In some examples, the first circumferential profile C1 of the first configuration 2010 is sized for a child or a young adult.

In some examples, the second circumferential profile C2 of the second configuration 2020 is sized for a young adult or an adult.

In some examples, the expandable thermoformed conduit 1902 is configured as a right ventricle to pulmonary artery (RV-PA) conduit.

### Additional Example 5: Expandable Conduit Having Pleated or Folded Regions

In some examples, the expandable conduit device as described in any of the previously described examples, may also include pleated or folded regions to accommodate an increase in the circumferential profile of the thermoformed conduit.

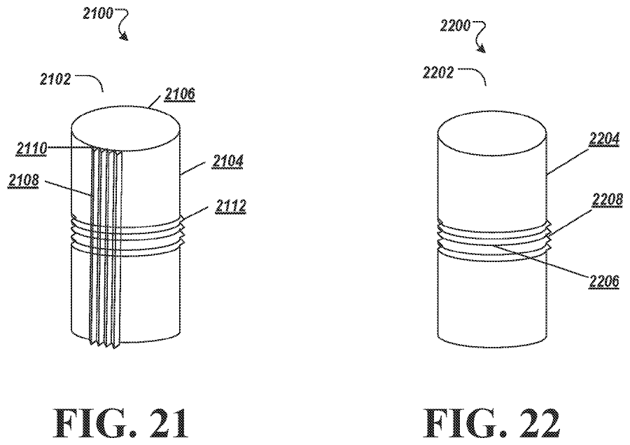

An example expandable conduit device 2100 is illustrated in FIG. 21. The expandable conduit device 2100 includes a thermoformed conduit 2102 having a length defined by a longitudinal profile 2104 of the thermoformed conduit 2102 and one or more circumferential profile, including a first circumferential profile 2106 defined along a portion of the longitudinal profile 2104 (ingress, egress, interchannel).

In some examples, as shown in FIG. 21, the expandable thermoformed conduit 2102 includes a circumferential pleated or folded region 2108 at a portion 2110 of the first circumferential profile 2106 when in the first configuration 2010, the circumferential pleated or folded region 2110 extending along the longitudinal profile or a portion thereof.

Another example expandable conduit device 2200 is illustrated in FIG. 22. The expandable conduit device 2200 includes a thermoformed conduit 2202 having a length defined by a longitudinal profile 2204 and includes a longitudinal pleated or folded region 2206 along the longitudinal profile 2204 or a portion 2208 thereof.

In some examples, as illustrated in FIG. 21, the expandable thermoformed conduit includes both a circumferential pleated or folded region and a longitudinal pleated or folded region. FIG. 21 shows such an example, wherein the expandable thermoformed conduit 2102 includes both a circumferential pleated or folded region 2108 and a longitudinal pleated or folded region 2112 similar to the region 2206 of FIG. 21.

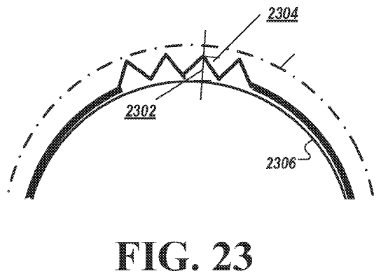

FIG. 23 further illustrates an expandable thermoformed conduit having circumferential pleated or folded regions. Specifically, FIG. 23 shows the circumferential profile of the expandable thermoformed conduit decreasing upon formation of a circumferential pleated or folded region. In the example shown in FIG. 23, the pleated or folded regions may be geometrically defined by a height parameter 2302 and an angle parameter 2304 to define a second circumferential profile, i.e., outer diameter, for the conduit (100). The height parameter 2302 can be defined from the inner radial position 2306 of the conduit to ensure a smooth internal surface that would contact vascular material or fluid.

In the example shown in FIG. 23, the pleated or folded region includes one or more individual pleats or folds in the expandable thermoformed conduit. Each individual pleat or fold defines a pleat or fold face having an edge that deviates from the shape of the circumferential profile before the formation of the pleated or folded region. Each edge has an edge distance. In some examples, the expandable thermoformed conduit has more than one individual pleat or fold. In such examples, adjacent faces (or edges) are offset at an offset angle.

During fabrication, individual pleats or folds are introduced at a circumferential pleated or folded region, thereby reducing the circumferential profile. Adjustment of edge distances and/or offset angles causes a corresponding change in the circumferential area defined by the circumferential profile. During fabrication, the circumferential area is reduced in preparation for implantation. After implantation, the expandable thermoformed conduit may be expanded such that the circumferential area is increased from the first configuration 2010 to the second configuration 2020. Such expansion results from a relative increase in offset angles. Analogous principles apply to the longitudinal pleats or folded regions. However, adjustments to the edge distances and/or offset angles of the individual longitudinal pleats or folds results in a relative increase or decrease in the length of the expandable thermoformed conduit such that the first end moves relative to the second end.

### Additional Example 6: Expandable Conduit Having Reinforcing Structures

In some examples, the expandable conduit device as described in any of the previously described examples may also include reinforcing structures.

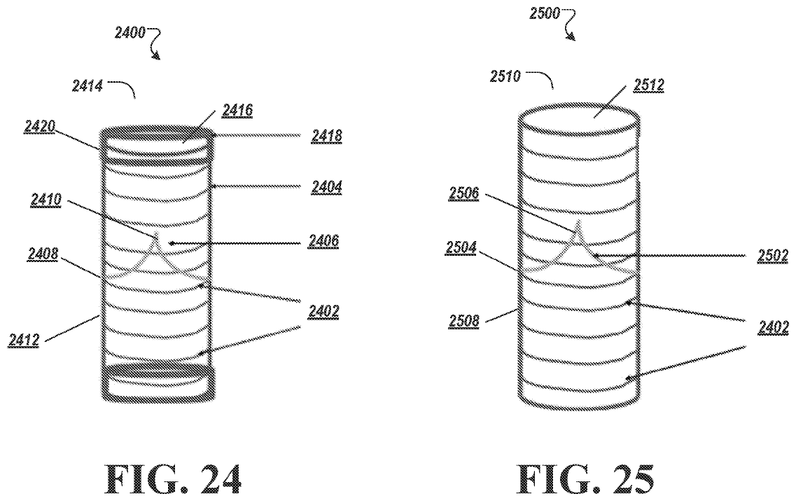

An example expandable conduit device 2400 is illustrated in FIG. 24. The expandable conduit device 2400 comprises a reinforcing structure 2402 disposed within the thermoformed conduit 2404. In some examples, the reinforcing structure 2402 between the inner and outer layers of the thermoformed conduit 2404.

In some examples, the reinforcing structure 2402 includes a stent-shaped structure comprising at least one strut.

In some examples, the reinforcing structure 2402 includes a metallic composite or alloy.

In some examples, the reinforcing structure 2402 includes a coil-shaped structure. The coil-shaped structure may include a continuous coil, as shown in FIG. 24, or may consist of a series of discrete rings spaced apart.

In some examples, the reinforcing structure 2402 includes a polymer or a thermoplastic material.

### Additional Example 7: Expandable Conduit Device Having Valves

In some examples, the expandable conduit device, as described in any of the previously described examples, may also include a valve. Such an expandable conduit device may accommodate somatic growth in pediatric patients who require right ventricular outflow tract (RVOT) reconstruction.

An example expandable conduit device is illustrated in FIG. 24. The expandable conduit device 2400 includes two or more leaflets 2406 configured to operate as a valve by moving collectively between an open state and a closed state. The two or more leaflets each include a first end 2408 and a second end 2410 and define a curved length therebetween. The first end 2410 of the two or more leaflets 2406 are disposed adjacent to an inner wall 2412 of the expandable thermoformed conduit 2414. The second end 2408 of the two or more leaflets 2406 extends into the channel 2416 such that they are disposed away from the inner wall 2412 of the expandable thermoformed conduit 2414.

In the open state, a space exists between the second ends 2410 of the two or more leaflets 2406 so as to permit the flow of fluid therethrough. In the closed state, the second end 2410 of at least one leaflet 2406 contacts the second ends 2410 of the other leaflets 2406 so as to reduce or prevent the flow of fluid therethrough. At least one of the two or more leaflets 2406 is sized to (i) form the closed state in connection with the other leaflets 2406.

In some examples, the expandable conduit device 2400 includes one or more sewable rings, including a first sewable ring 2418. The first sewable ring 2418 is coupled to a circumferential portion of a first end 2420 of the expandable conduit device 2400.

A sewable ring allows for the device to be sutured to its corresponding region of the subject's heart in order to position it in an operative position. In some examples, first and second sewable rings are formed from a thermoplastic material or a fabric.

### Expandable Valve

In some examples, the valve formed by the two or more leaflets can itself expand to accommodate an increase in the circumferential profile of the thermoformed conduit.

An example expandable conduit device 2500 is illustrated in FIG. 25. The expandable conduit device 2500 includes two or more leaflets 2506 configured to operate as a valve by moving collectively between an open state and a closed state. The two or more leaflets 2502 each include a first end 2504 and a second end 2506 and define a curved length therebetween. The first ends 2504 of the two or more leaflets 2502 are disposed adjacent to an inner wall 2508 of the expandable thermoformed conduit 2510. The second ends 2506 of the two or more leaflets 2502 extend into the channel 2512 such that they are disposed away from the inner wall 2508 of the expandable thermoformed conduit 2510.

The expandable conduit device 2500 comprises a reinforcing structure 2402 disposed within the thermoformed conduit 2510. In some examples, the reinforcing structure 2402 between the inner and outer layers of the thermoformed conduit 2510.

In some examples, the reinforcing structure 2402 includes a stent-shaped structure comprising at least one strut.

In some examples, the reinforcing structure 2402 includes a metallic composite or alloy.

In the open state, a space exists between the second ends 2506 of the two or more leaflets 2502 so as to permit the flow of fluid therethrough. In the closed state, the second end 2506 of at least one leaflet 2502 contacts the second end 2506 of the other leaflets 2502 so as to reduce or prevent the flow of fluid therethrough. At least one of the two or more leaflets 2502 is sized to (i) form the closed state in connection with the other leaflets 2502 in the first configuration 2010 and (ii) form the closed state in connection with other leaflets in the second configuration 2020.

Expandable Method 1: Expandable Conduit (without Valve) and Expandable Valve

The expandable conduit device 2500 shown in FIG. 25 includes at least two components—an expandable thermoformed conduit 2510 and two or more leaflets 2502 operating as an expandable valve. The expandable thermoformed conduit 2510 was expanded using a transcatheter balloon dilator. The graft material, HA-LLDPE, was expanded in its plastic state to achieve permanent deformation resulting in an increase in its diameter.

The expandable valve was inserted into the graft before surgical implantation and had the ability to increase its annulus diameter while maintaining excellent hemodynamic performance for the varying physiological loads due to somatic growth. Thus, the expandable thermoformed conduit and expandable valve worked together to accommodate patient growth and served as a durable and low-cost device option to reduce the number of surgical reinterventions.

Expandable Method 2: Expandable Valved Conduit (with Non-Expandable Valve) and Transcatheter Pulmonary Valve (TPV)

The device 2600, shown in FIG. 26, has included at least two components—an expandable thermoformed conduit 2620 containing leaflets 2602 and a transcatheter pulmonary valve (TPV) 2604. The expandable thermoformed conduit 2620 had its own leaflets 2606, allowing it to perform as a valved conduit before expansion. After expansion, using a transcatheter balloon dilator, the leaflets 2602 did not have the required hemodynamic performance because they were not designed to accommodate growth. Hence, a TPV 2604, designed specifically for this expandable thermoformed conduit, was deployed after expansion. Current clinical practices involved deploying a commercial TPV into a stenosed pulmonary valved conduit to restore its function.

The TPV 2604 had leaflets 2606 made from HA-LLDPE, which provided good hemodynamics and a low-cost, durable alternative to current tissue-based TPVs. In some examples, the TPV 2604 can include a stent formed of metallic or polymeric materials. In such examples, the stent is crimped for insertion and re-expanded after transcatheter deployment. In this way, an expandable thermoformed conduit and post-expansion TPV can work together to accommodate patient growth and helped reduce the number of surgical reinterventions.

The expandable conduit device 2600 comprises a reinforcing structure 2402 disposed within the thermoformed conduit 2620. In some examples, the reinforcing structure 2402 between inner and outer layers of the thermoformed conduit 2620.

In some examples, the reinforcing structure 2402 includes a stent-shaped structure comprising at least one strut.

In some examples, the reinforcing structure 2402 includes a metallic composite or alloy.

### Technical Description for Expandable Graft

The expandable thermoformed conduit was made from HA-LLDPE and was permanently deformed with the help of a balloon dilator. To increase the conduit's size circumferentially (e.g., its diameter) and longitudinally/axially (e.g., its length), extra HA-LLDPE material was incorporated in the design using accordion-like folds. Throughout the length of the conduit, a mesh-like reinforcement that was flexible (allowed for curved conduits) was provided. The mesh was metallic or polymeric, depending on the design and manufacturing techniques. This mesh was embedded/sandwiched between the two LLDPE layers during thermoforming. At strategic points in the axial direction and at the anastomoses, the mash was connected to locking members that helped control the expansion and prevented over-expansion whilst providing structural support to the conduit. These locking members consisted of sliding mechanisms that can slide over each other during expansion and then interlocked at a desired point once the required expansion was achieved.

The mesh, along with the locking mechanism at the anastomoses, was covered externally by medical-grade fabrics or elastic and soft materials to create sewing rings. These sewing rings were used to suture the conduit to the right ventricle and pulmonary artery to restore blood flow to the lungs.

### Additional Example 8: Example Device and Method of Using an Expandable Valved Conduit

An example method is disclosed to fabricate a valved conduit for heart valve applications. The valved conduit can be made of any group of thermoplastics, including but not limited to polyethylene, polycarbonates, polyvinyl chlorides, polyesters, polyether, polytetrafluoroethylenes, polyamides, polystyrenes, thermoplastic polyurethane, and polypropylene. The thermoplastic conduit may be biocompatible using processes and operations such as those described in US Patent Publication No. 20180305528 that incorporates hyaluronic acid (HA) into polyethylene (the product is referred to as HA-PE).

This technique has demonstrated increased blood compatibility, reduced calcification, and prevented immune rejection of PE heart valve leaflets in-vitro and in-vivo. While the HA-PE material is suited for use as a valved conduit, the conduit itself must be sutured to native tissue. The fabric may be incorporated for suturing in at the ends of the conduit. The fabric can be any textile material but is not limited to nylons, polyesters, kinds of cotton, and celluloses.

An example sewing ring incorporated into PE (e.g., polyethylene terephthalate (PET) may be incorporated or integrated into the conduit. Polyethylene terephthalate (PET) is a known material used for surgical attachments (Alfieris, 2016; Rüffer, 2912; Brown, 2001). By developing HA-PE as a valved conduit with PET sewing rings, the example process and device may provide patients with CHD's a safer, more effective, and more cost-effective option for treatment. The valve portion of the conduit may also be shaped based on a mold used in the thermoforming process in which the curvature and geometry of the valve can be readily adjusted.

Production of pilot RV-PA Conduit

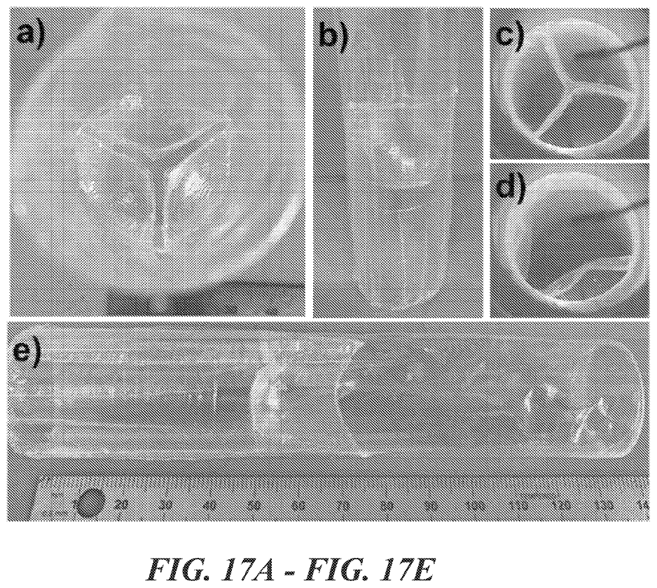

FIG. 16A-FIG. 16F are diagrams showing the manner in which the valved conduit is shaped using thermoforming. FIG. 17A-FIG. 17E demonstrate an example functioning prototype of the valved conduit.

In this example, the conduit is made of a linear low-density polyethylene (LLDPE). LLDPE cannot be sewn directly, presenting an issue for surgical attachment; however, the example method employed polyethylene terephthalate (PET), which can be incorporated into the LLDPE. PET (a.k.a. Dacron®) is known to have excellent handling characteristics and may be readily sutured without leakage leading to its use as a sewing ring in many cardiovascular applications, including right ventricle to pulmonary artery (RV-PA) conduits (Alfieris, 2016; Rüffer, 2912; Brown, 2001).

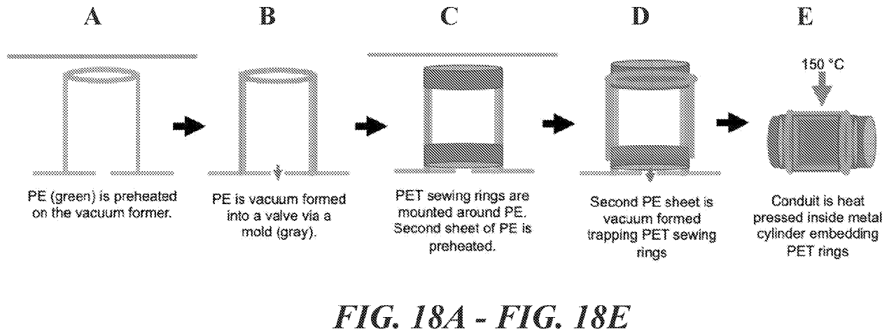

PET sewing rings may be incorporated into the LLDPE conduit providing a method of attachment to both the native heart tissue and the pulmonary artery. The diagram in FIG. 18A-FIG. 18E demonstrates the process.

It should be noted this is a slightly modified version of the process shown in FIG. 16A-FIG. 16F.

Briefly, the PET sewing cuffs may be incorporated at the conduit openings in the following manner. (1) LLDPE may be thermoformed into a cylinder. (2) PET sewing rings may be slipped around the LLDPE cylinder at the inlet and outlet. (3) A second sheet of LLDPE may be thermoformed around the first LLDPE cylinder (with PET sewing rings), trapping the rings between the LLDPE sheets. A final melt pressing step (as seen in the study results) may be used to embed the PET into to LLDPE and weld the LLDPE sheets together (FIG. 18A-FIG. 18E). PET has a melting point of 260° C., thus, it would tolerate LLDPE bonding.

### EXPERIMENTAL RESULTS AND EXAMPLES

Two valved conduits of different lengths were tested in a right heart simulator. The conduits were made of LLDPE film in the thermoforming process shown in FIG. 16A-FIG. 16F. The simulator was programmed for an in-vitro under pulsatile pulmonary flow conditions (8/25 mm Hg diastolic/peak systolic pressure), heart rate (60 bpm), and cardiac output (5 L/min). A working fluid of 60/40 water to glycerin (99% pure glycerin) was used to provide density and kinematic viscosity comparable to blood at 1060 kg/m³ and 3.5·10⁻⁶ m²/s, respectively. Pressure gradient, as well as flow waveforms, were collected at a sampling frequency of 100 Hz for 60 consecutive cardiac cycles. From these data, the mean transvalvular pressure gradient (ΔP), peak transvalvular pressure gradient (peak ΔP), effective orifice area (EOA), and a regurgitant fraction (RF) were computed for each of the valved conduits. The results are shown in Table 1, above, and demonstrate the prototype functions and have the potential to be an effective RVOT replacement.

To determine if the PET rings could be thermally incorporated into PE, PET fabric was melt pressed between linear low-density polyethylene sheets (LLDPE), and dog bones specimens taken from the LLDPE/PET construct were pulled to failure at a crosshead speed of 25 mm/min.

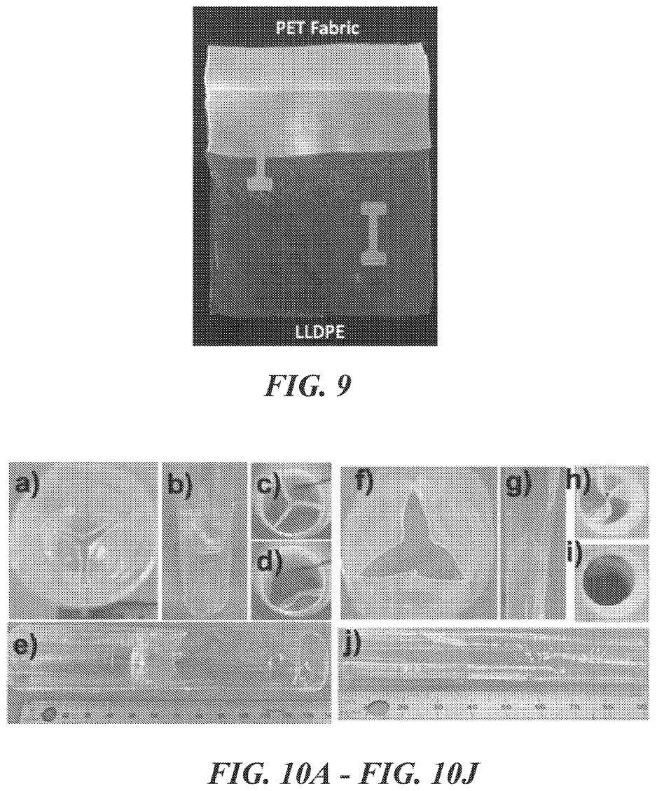

FIG. 9 shows the construct and has representative dog bones shown where samples were punched.

The results of the interface (green dog bone) were compared to the control (red dog bone). The LLDPE/PET interface (green dog bone) shown in FIG. 4 was mechanically similar to the control LLDPE (red dog bone). The interface had a modulus, yield strength, and ultimate tensile strength of 82.63±18.74 MPa, 6.71±1.34 MPa, 8.66±1.36 MPa, respectively, compared to the LLDPE with 70.45±7.40 MPa, 6.97=0.99 MPa, 10.65±2.51 MPa, respectively. This indicates that the thermoforming melt flow process by which PET is embedded in LLDPE will provide a satisfactory method to incorporate a PET sewing ring for the conduit anastomoses; even though the green interface is slightly weaker, it should withstand the low pulmonary pressures (35 mmHg or 4.7*10⁻³ MPa) (Momenah, 2009).

### Discussion

Congenital heart defects such as aortic valve stenosis and ventricular outflow tract dysfunctions often require specialized devices in pediatric patients (Yuan, 2008; CDC, 2020; da Costa, 2017; DiBardino, 2014; Kan, 2018; Shinkawa, 2010; Stelzer, 2011). Patients born with critical congenital heart defects (CHDs) such as but not limited to hypoplastic left heart syndrome, pulmonary atresia, truncus arteriosus, congenital aortic stenosis, and transposition of the great arteries require valved conduits for right ventricular outflow tract reconstruction. These CHDs require surgical intervention to replace the right ventricular outflow tract (RVOT), and currently available prostheses such as homografts, xenografts, and expanded polytetrafluoroethylene conduits have well-known issues with cost, availability, calcification, immune rejection, endocarditis, and stenosis (Ong, 2013; Carreon, 2019; Baskett, 1996; Martin, 2018; Alfieris, 2016).

With CHD and other defects such as aortic and pulmonary stenosis, patients often require heart valve replacements. Optimal valve substitutes would constitute a valve that is the right patient size, has the potential to grow, has minimal susceptibility to thrombosis, and demonstrates excellent prosthesis longevity. Currently, the standard of care for pediatric valve replacement is mechanical prosthesis or a smaller-sized adult tissue prosthesis that requires lifelong anticoagulation or risks limited longevity due to increasing patient-prosthesis mismatch and is not intended for use in pediatric patients (Carreon, 2019; Baskett, 1996; Alfieris, 2016; Yong, 2015; Hawkins, 2000). Common complications involving off-label pediatric valves include valve dislodgement, stent fracturing, recurrent stenosis, and regurgitation (Rüffer, 2012; Hill, 2016; Cheatham, 2015; Kenny, 2018).

### Additional Example 9: Method of Operating an Expandable Conduit Device

In some examples, the expandable conduit device, as described in any of the previously described examples, may be used in a surgical operation.

Provided herein are methods of operating an expandable conduit device. The method includes providing an expandable thermoformed conduit comprising a low melting point thermoplastic material suitable for thermoforming, the thermoformed conduit including a first end and a second end, and defining a channel therebetween. The conduit device is configured to be surgically embedded into a subject's heart. The expandable thermoformed conduit is configured to have a first configuration having a first circumferential profile at a first position in the channel and a second configuration having a second circumferential profile at the first position, wherein the second circumferential profile has a larger cross-sectional area than the first circumferential profile.

The expandable conduit device may then be implanted while the expandable thermoformed conduit is in the first configuration. Implanting the expandable conduit device may include locating and removing a segment of diseased tissue or identifying the site of otherwise deficient anatomy. The expandable conduit device is then implanted into the subject's anatomy, such as at the heart or at a blood vessel. Once positioned, the expandable thermoformed conduit may be mechanically induced to expand from the first configuration to the second configuration.

Mechanically inducing the expansion may include any of the methods described in any of the previous examples. This may include coupling an expandable member to a catheter or other surgical instrument and introducing it into a subject intravenously. The expandable member may then be positioned in a position within the channel of the expandable thermoformed conduit by advancing the expandable member through the subject's vasculature. Once positioned, the expandable member is expanded such that it contacts an inner wall of the expandable thermoformed conduit and exerts an outwardly directed force thereupon so as to adjust the cross-sectional area of the expandable thermoformed conduit at the first position from the first circumferential profile to the second circumferential profile.

In some examples, the expandable thermoformed conduit is configured as a transcatheter pulmonary valve.

In some examples, the expandable thermoformed conduit is configured as a right ventricle to pulmonary artery (RV-PA) conduit.

In some examples, the first configuration is sized for a child or a young adult.

In some examples, the second configuration is sized for a young adult or an adult.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The following patents, applications and publications as listed below and throughout this document are hereby incorporated by reference in their entirety herein.

### REFERENCES

GBD 2017 Congenital Heart Disease Collaborators, Lancet Child Adolesc Health. 2020 March; 4 (3): 185-200.
Corno, A. F. et al, Ann. Thorac. Surg. 78, 1382-1388 (2004).
Ootaki, Y. et al, Ann. Thorac. Surg. 105, 843-850 (2018).
Yamamoto, Y. et al, Semin. Thorac. Cardiovasc. Surg. 32, 990-999 (2020).
Prodan, Z. et al, Semin. Thorac. Cardiovasc. Surg. (2021)
Carreon, C. K. et al, J Thorac Cardiovasc Surg. 157, 342-350 (2019)
Prawel, D. A. et al, Cardiovasc. Eng. Technol. 5, 70-81 (2014).
Heitkemper M. et. al, Novel Hyaluronan Enhanced Polymeric Transcatheter Aortic Valve Replacement: A proof of concept. in (2018).
Paytoncu, Ş, J. Cardiovasc. Med. 7, 126-132 (2019).
Sfyridis, P. G. et al., J. Cardiol. 52, 501-508 (2011).
Carreon, C. K. et al, J Thorac Cardiovasc Surg. 157, 342-350 (2019)
Manavitehrani, I., et al. Cardiovasc Eng Tech 10, 205-215 (2019).
Guariento, A. et al, J Thorac Cardiovasc Surg. 163, 224-236 (2021)
Prawel, D. A. et al, Cardiovasc. Eng. Technol. 5, 70-81 (2014).
Sfyridis, P. G. et al., J. Cardiol. 52, 501-508 (2011).
Homann, M. et al. Reconstruction of the RVOT with valved biological conduits: 25 years experience with allografts and xenografts*. Eur. J. Cardiothorac. Surg. 17, 624-630 (2000).
Corno, A. F. et al. Bovine Valved Xenograft in Pulmonary Position: Medium-Term Follow-Up With Excellent Hemodynamics and Freedom From Calcification. Ann. Thorac. Surg. 78, 1382-1388 (2004).
Ootaki, Y. et al. Medium-Term Outcomes After Implantation of Expanded Polytetrafluoroethylene Valved Conduit. Ann. Thorac. Surg. 105, 843-850 (2018).
Yamamoto, Y. et al. Histopathologic Analysis of Explanted Polytetrafluoroethylene-Valved Pulmonary Conduits. Semin. Thorac. Cardiovasc. Surg. 32, 990-999 (2020).
Prodan, Z. et al. Initial Clinical Trial of a Novel Pulmonary Valved Conduit. Semin. Thorac. Cardiovasc. Surg. (2021) doi:10.1053/j.semtcvs.2021.03.036.
James, S. P., Zhang, M. & Beauregard, G. Outer layer having entanglement of hydrophobic polymer host and hydrophilic polymer guest. (2010).
James, S. P. et al. Glycosaminoglycan and synthetic polymer materials for blood-contacting applications. (2015).
Prawel, D. A. et al. Hemocompatibility and Hemodynamics of Novel Hyaluronan-Polyethylene Materials for Flexible Heart Valve Leaflets. Cardiovasc. Eng. Technol. 5, 70-81 (2014).
Reitsma, S., Slaaf, D. W., Vink, H., van Zandvoort, M. A. M. J. & oude Egbrink, M. G. A. The endothelial glycocalyx: composition, functions, and visualization. Pflugers Arch. 454, 345-359 (2007).
Heitkemper M, Hatoum H, Simon-Walker R, Bui H, Popat K, James S, Cheatham J and Dasi L P. Novel Hyaluronan Enhanced Polymeric Transcatheter Aortic Valve Replacement: A proof of concept. in (2018).
Bui, H., Ishrat, A., James, S. & Dasi, L. P. Durability optimization of novel hyaluronic acid enhanced polymeric transcatheter aortic valves. J. Am. Coll. Cardiol. 77, 3236-3236 (2021).
James, S. P. & Zhang, M. Hyaluronan (HA) esterification via acylation technique for moldable devices. (2010).
14:00-17:00. ISO 5840-1:2021./SO https://www.iso.org/cms/render/live/en/sites/isoorg/contents/data/standard/07/70/77033.html.
Belli, E. et al. The Performance of Hancock Porcine-Valved Dacron Conduit for Right Ventricular Outflow Tract Reconstruction. Ann. Thorac. Surg. 89, 152-158 (2010).
Brown, J. W., Ruzmetov, M., Rodefeld, M. D., Vijay, P. & Turrentine, M. W. Right ventricular outflow tract reconstruction with an allograft conduit in non-ross patients: risk factors for allograft dysfunction and failure. Ann. Thorac. Surg. 80, 655-663; discussion 663-664 (2005).
Yuan, S.-M., Mishaly, D., Shinfeld, A. & Raanani, E. Right ventricular outflow tract reconstruction: valved conduit of choice and clinical outcomes. J. Cardiovasc. Med. 9, 327-337 (2008).
Mery, C. M. et al. Risk factors for development of endocarditis and reintervention in patients undergoing right ventricle to pulmonary artery valved conduit placement. J. Thorac. Cardiovasc. Surg. 151, 432-439, 441.e1-2 (2016).
Morales, D. L. et al. A Novel Restorative Pulmonary Valve Conduit: Early Outcomes of Two Clinical Trials.
Front. Cardiovasc. Med. 7, 367 (2021).
Capelli, C. et al. Patient-specific simulations for planning treatment in congenital heart disease. Interface Focus 8, 20170021 (2018).
CDC. Critical Congenital Heart Defects I CDC. Centers for Disease Control and Prevention https://www.cdc.gov/ncbddd/heartdefects/cchd-facts.html (2020).
Stefanescu Schmidt, A. C. et al. National Trends in Hospitalizations of Adults with Tetralogy of Fallot. Am. J. Cardiol. 118, 906-911 (2016).
Brown, J. W. et al. Rastelli Operation for Transposition of the Great Arteries With Ventricular Septal Defect and Pulmonary Stenosis. Ann. Thorac. Surg. 91, 188-194 (2011).
Mastropietro, C. W. et al. Characteristics and operative outcomes for children undergoing repair of truncus arteriosus: A contemporary multicenter analysis. J. Thorac. Cardiovasc. Surg. 157, 2386-2398.e4 (2019).
Brown, J. W., Ruzmetov, M., Okada, Y., Vijay, P. & Turrentine, M. W. Truncus arteriosus repair: outcomes, risk factors, reoperation and management. Eur. J. Cardiothorac. Surg. 20, 221-227 (2001).
Elder, R. W. et al. Outcomes of the infant Ross procedure for congenital aortic stenosis followed into adolescence. J. Thorac. Cardiovasc. Surg. 145, 1504-1511 (2013).
Yasukawa, T. et al. Interstage management of pulmonary blood flow after the Norwood procedure with right ventricle-to-pulmonary artery conduit. Eur. J. Cardiothorac. Surg. 58, 551-558 (2020).
da Costa, F. D. A. et al. Decellularized Allografts for Right Ventricular Outflow Tract Reconstruction in Children. World J. Pediatr. Congenit. Heart Surg. 8, 605-612 (2017).
DiBardino, D. J. & Jacobs, J. P. Current Readings: Long-Term Management of Patients Undergoing Successful Pediatric Cardiac Surgery. Semin. Thorac. Cardiovasc. Surg. 26, 132-144 (2014).
Kan, C.-D. et al. Applicability of handmade expanded polytetrafluoroethylene trileaflet-valved conduits for pulmonary valve reconstruction: An ex vivo and in vivo study. J. Thorac. Cardiovasc. Surg. 155, 765-774.e3 (2018).
Shinkawa, T., Bove, E. L., Hirsch, J. C., Devaney, E. J. & Ohye, R. G. Intermediate-Term Results of the Ross Procedure in Neonates and Infants. Ann. Thorac. Surg. 89, 1827-1832 (2010).
Stelzer, P. The Ross Procedure: State of the Art 2011. Semin. Thorac. Cardiovasc. Surg. 23, 115-123 (2011).
Choi, K. H. et al. Late results of right ventricular outflow tract reconstruction with a bicuspid expanded polytetrafluoroethylene valved conduit. J. Card. Surg. 33, 36-40 (2018).
Suzuki, I. et al. Engineering analysis of the effects of bulging sinuses in a newly designed pediatric pulmonary heart valve on hemodynamic function. J. Artif. Organs Off. J. Jpn. Soc. Artif. Organs 15, 49-56 (2012).
Bonetti, A., Marchini, M. & Ortolani, F. Ectopic mineralization in heart valves: new insights from in vivo and in vitro procalcific models and promising perspectives on noncalcifiable bioengineered valves. J. Thorac. Dis. 11, 2126-2143 (2019).
Ong, K. et al. Right Ventricle to Pulmonary Artery Conduit Reoperations in Patients With Tetralogy of Fallot or Pulmonary Atresia Associated With Ventricular Septal Defect. Am. J. Cardiol. 111, 1638-1643 (2013).
Carreon, C. K. et al. Pathology of valved venous homografts used as right ventricle-to-pulmonary artery conduits in congenital heart disease surgery. J. Thorac. Cardiovasc. Surg. 157, 342-350.e3 (2019).
Christenson, J. T., Vala, D., Sierra, J., Beghetti, M. & Kalangos, A. Blood group incompatibility and accelerated homograft fibrocalcifications. J. Thorac. Cardiovasc. Surg. 127, 242-250 (2004).
Reinhartz Olaf et al. Homograft Valved Right Ventricle to Pulmonary Artery Conduit as a Modification of the Norwood Procedure. Circulation 114, I-594 (2006).
Christenson, J. T. et al. Homografts and Xenografts for Right Ventricular Outflow Tract Reconstruction: Long-Term Results. Ann. Thorac. Surg. 90, 1287-1293 (2010).
Baskett, R. J., Ross, D. B., Nanton, M. A. & Murphy, D. A. Factors in the early failure of cryopreserved homograft pulmonary valves in children: Preserved immunogenicity? J. Thorac. Cardiovasc. Surg. 112, 1170-1179 (1996).
Hawkins, J. A. et al. Class I and class II anti-HLA antibodies after implantation of cryopreserved allograft material in pediatric patients. J. Thorac. Cardiovasc. Surg. 119, 324-330 (2000).
Martin Mary Hunt et al. Safety and Feasibility of Melody Transcatheter Pulmonary Valve Replacement in the Native Right Ventricular Outflow Tract. JACC Cardiovasc. Interv. 11, 1642-1650 (2018).
Alfieris, G. M., Swartz, M. F., Lehoux, J. & Bove, E. L. Long-Term Survival and Freedom from Reoperation After Placement of a Pulmonary Xenograft Valved Conduit. Ann. Thorac. Surg. 102, 602-607 (2016).
Dunne, B. et al. The freestyle valve as a right ventricle to pulmonary artery conduit. A systematic review and meta-analysis. Heart Lung Vessels 7, 304-310 (2015).
Dijck, I. V. et al. Infective endocarditis of a transcatheter pulmonary valve in comparison with surgical implants. Heart 101, 788-793 (2015).
Yong, M. S. et al. Medium-term outcomes of bovine jugular vein graft and homograft conduits in children. ANZ J. Surg. 85, 381-385 (2015).
Peivandi, A. D. et al. Elastica degeneration and intimal hyperplasia lead to Contegra® conduit failure. Eur. J. Cardiothorac. Surg. 56, 1154-1161 (2019).
Nichay, N. R. et al. In search of the best xenogeneic material for a paediatric conduit: an analysis of clinical datat. Interact. Cardiovasc. Thorac. Surg. 27, 34-41 (2018).
Miyazaki, T. et al. Expanded polytetrafluoroethylene valved conduit and patch with bulging sinuses in right ventricular outflow tract reconstruction. J. Thorac. Cardiovasc. Surg. 134, 327-332 (2007).
Bennink, G. et al. A novel restorative pulmonary valved conduit in a chronic sheep model: Mid-term hemodynamic function and histologic assessment. J. Thorac. Cardiovasc. Surg. 155, 2591-2601.e3 (2018).
Flameng, W. et al. Factors influencing calcification of cardiac bioprostheses in adolescent sheep. J. Thorac. Cardiovasc. Surg. 132, 89-98 (2006).
Bui, H., Harris, K., Li, E., Prawel, D. & James, S. The Development and Fabrication of Vapor Crosslinked Hyaluronan-Polyethylene Interpenetrating Polymer Network as a Biomaterial. ACS Appl. Mater. Interfaces 11, (2019).
Zhang, M., King, R., Hanes, M. & James, S. P. A novel ultra high molecular weight polyethylene-hyaluronan microcomposite for use in total joint replacements. I. Synthesis and physical/chemical characterization. J. Biomed. Mater. Res. A 78A, 86-96 (2006).
Perin, E. C., Miller, L. W., Taylor, D. A. & Willerson, J. T. Organogenesis. in Stem Cell and Gene Therapy for Cardiovascular Disease 349-373 (Academic Press, 2015).
Momenah Tarek S. et al. Extended Application of Percutaneous Pulmonary Valve Implantation. J. Am. Coll. Cardiol. 53, 1859-1863 (2009).
Sfyridis, P. G. et al. The Contegra® valved heterograft conduit for right ventricular outflow tract reconstruction: a reliable solution. Hell. J. Cardiol. HJC Hell. Kardiologike Epitheorese 52, 501-508 (2011).
Purohit, M., Kitchiner, D. & Pozzi, M. Contegra bovine jugular vein right ventricle to pulmonary artery conduit in Ross procedure. Ann. Thorac. Surg. 77, 1707-1710 (2004).
Heitkemper, M. et al. Modeling risk of coronary obstruction during transcatheter aortic valve replacement. J. Thorac. Cardiovasc. Surg. 159, 829-838.e3 (2020).
Bui, H., James, S. & Dasi, L. TCT-326 In Vitro Performance of Thermoformed Polymeric Transcatheter Heart Valves. in Journal of the American College of Cardiology vol. 78 B133-B133 (American College of Cardiology Foundation, 2021).
Bui, H. T., James, S. P. & DASI, L. P. Design and Fabrication of a Novel Polymeric Transcatheter Aortic Valve. in (2021).
Sadri, V., Madukauwa-David, I. D. & Yoganathan, A. P. In vitro evaluation of a new aortic valved conduit. J. Thorac. Cardiovasc. Surg. 161, 581-590.e6 (2021).
Thubrikar, M. The aortic valve. (CRC Press, 1990).
Sluysmans, T. & Colan, S. D. Theoretical and empirical derivation of cardiovascular allometric relationships in children. J. Appl. Physiol. 99, 445-457 (2005).
Paytoncu, $. The Aortic Annulus, Sinus of Valsalva, Ascending Aorta and Z-score Values in Healthy Children. E-J. Cardiovasc. Med. 7, 126-132 (2019).
Hatoum, H., Lilly, S., Maureira, P., Crestanello, J. & Prasad Dasi, L. Sinus Hemodynamics After Transcatheter Aortic Valve in Transcatheter Aortic Valve. Ann. Thorac. Surg. 110, 1348-1356 (2020).
Moore, B. L. Influence of anatomic valve conditions and coronary flow on aortic sinus hemodynamics. (Colorado State University, 2014).
Morray Brian H. et al. Multicenter Experience Evaluating Transcatheter Pulmonary Valve Replacement in Bovine Jugular Vein (Contegra) Right Ventricle to Pulmonary Artery Conduits. Circ. Cardiovasc. Interv. 10, e004914 (2017).
Leo, H. L., Dasi, L. P., Carberry, J., Simon, H. A. & Yoganathan, A. P. Fluid dynamic assessment of three polymeric heart valves using particle image velocimetry. Ann. Biomed. Eng. 34, 936-952 (2006).
Dasi, L. P., Ge, L., Simon, H. A., Sotiropoulos, F. & Yoganathan, A. P. Vorticity dynamics of a bileaflet mechanical heart valve in an axisymmetric aorta. Phys. Fluids 19, 067105 (2007).
Dasi, L. P., Murphy, D. W., Glezer, A. & Yoganathan, A. P. Passive flow control of bileaflet mechanical heart valve leakage flow. J. Biomech. 41, 1166-1173 (2008).
Hartley, R., Zisserman, A. Multiple View Geometry in Computer Vision. (Cambridge University Press, 2003).
Smith, D. B., Sacks, M. S., Vorp, D. A. & Thornton, M. Surface geometric analysis of anatomic structures using biquintic finite element interpolation. Ann. Biomed. Eng. 28, 598-611 (2000).
Yoganathan, A. P., He, Z. & Casey Jones, S. Fluid mechanics of heart valves. Annu. Rev. Biomed. Eng. 6, 331-362 (2004).
Yoganathan, A. P. et al. A new paradigm for obtaining marketing approval for pediatric-sized prosthetic heart valves. J. Thorac. Cardiovasc. Surg. 146, 879-886 (2013).
Ge, L., Dasi, L. P., Sotiropoulos, F. & Yoganathan, A. P. Characterization of hemodynamic forces induced by mechanical heart valves: Reynolds vs. viscous stresses. Ann. Biomed. Eng. 36, 276-297 (2008).
Heitkemper, M., Hatoum, H. & Dasi, L. P. In Vitro Hemodynamic Assessment of a Novel Polymeric Transcatheter Aortic Valve. J. Mech. Behav. Biomed. Mater. 98, 163-171 (2019).
Schreiber, C. et al. Ring-Enforced Right Ventricle-to-Pulmonary Artery Conduit in Norwood Stage I Reduces Proximal Conduit Stenosis. Ann. Thorac. Surg. 88, 1541-1545 (2009).
Bentham, J. R., Baird, C. W., Porras, D. P., Rathod, R. H. & Marshall, A. C. A reinforced right-ventricle-to-pulmonary-artery conduit for the stage-1 Norwood procedure improves pulmonary artery growth. J. Thorac. Cardiovasc. Surg. 149, 1502-1508.e1 (2015).
Paul, D. M. et al. Suture Dehiscence in the Tricuspid Annulus: An Ex Vivo Analysis of Tissue Strength and Composition. Ann. Thorac. Surg. 104, 820-826 (2017).
Kiesendahl, N., Schmitz, C., Menne, M., Schmitz-Rode, T. & Steinseifer, U. In Vitro Calcification of Bioprosthetic Heart Valves: Test Fluid Validation on Prosthetic Material Samples. Ann. Biomed. Eng. (2020) doi:10.1007/s10439-020-02618-6.
Boloori Zadeh, P., Corbett, S. C. & Nayeb-Hashemi, H. In-vitro calcification study of polyurethane heart valves. Mater. Sci. Eng. C 35, 335-340 (2014).
Herijgers, P. et al. Valved jugular vein segments for right ventricular outflow tract reconstruction in young sheep. J. Thorac. Cardiovasc. Surg. 124, 798-805 (2002).
Azakie, A., Carney, J. P., Lahti, M. T., Moklyak, Y. & Bianco, R. W. Anisotropic Polytetrafluoroethylene Cardiovascular Conduits Spontaneously Expand in a Growing Lamb Model. J. Invest. Surg. 0, 1-7 (2020).
Yuan, S.-M., Mishaly, D., Shinfeld, A. & Raanani, E. Right ventricular outflow tract reconstruction: valved conduit of choice and clinical outcomes. J. Cardiovasc. Med. 9, 327-337 (2008).
CDC. Critical Congenital Heart Defects CDC. Centers for Disease Control and Prevention https://www.cdc.gov/ncbddd/heartdefects/cchd-facts.html (2020).
da Costa, F. D. A. et al. Decellularized Allografts for Right Ventricular Outflow Tract Reconstruction in Children. World J. Pediatr. Congenit. Heart Surg. 8, 605-612 (2017).
DiBardino, D. J. & Jacobs, J. P. Current Readings: Long-Term Management of Patients Undergoing Successful Pediatric Cardiac Surgery. Semin. Thorac. Cardiovasc. Surg. 26, 132-144 (2014).
Kan, C.-D. et al. Applicability of handmade expanded polytetrafluoroethylene trileaflet-valved conduits for pulmonary valve reconstruction: An ex vivo and in vivo study. J. Thorac. Cardiovasc. Surg. 155, 765-774.e3 (2018).
Shinkawa, T., Bove, E. L., Hirsch, J. C., Devaney, E. J. & Ohye, R. G. Intermediate-Term Results of the Ross Procedure in Neonates and Infants. Ann. Thorac. Surg. 89, 1827-1832 (2010).
Stelzer, P. The Ross Procedure: State of the Art 2011. Semin. Thorac. Cardiovasc. Surg. 23, 115-123 (2011).
Ong, K. et al. Right Ventricle to Pulmonary Artery Conduit Reoperations in Patients With Tetralogy of Fallot or Pulmonary Atresia Associated With Ventricular Septal Defect. Am. J. Cardiol. 111, 1638-1643 (2013).
Carreon, C. K. et al. Pathology of valved venous homografts used as right ventricle-to-pulmonary artery conduits in congenital heart disease surgery. J. Thorac. Cardiovasc. Surg. 157, 342-350.e3 (2019).
Baskett, R. J., Ross, D. B., Nanton, M. A. & Murphy, D. A. Factors in the early failure of cryopreserved homograft pulmonary valves in children: Preserved immunogenicity? J. Thorac. Cardiovasc. Surg. 112, 1170-1179 (1996).
Martin Mary Hunt et al. Safety and Feasibility of Melody Transcatheter Pulmonary Valve Replacement in the Native Right Ventricular Outflow Tract. JACC Cardiovasc. Interv. 11, 1642-1650 (2018).
Alfieris, G. M., Swartz, M. F., Lehoux, J. & Bove, E. L. Long-Term Survival and Freedom From Reoperation After Placement of a Pulmonary Xenograft Valved Conduit. Ann. Thorac. Surg. 102, 602-607 (2016).
Miyazaki, T. et al. Expanded polytetrafluoroethylene valved conduit and patch with bulging sinuses in right ventricular outflow tract reconstruction. J. Thorac. Cardiovasc. Surg. 134, 327-332 (2007).
Dijck, I. V. et al. Infective endocarditis of a transcatheter pulmonary valve in comparison with surgical implants. Heart 101, 788-793 (2015).
Dunne, B. et al. The freestyle valve as a right ventricle to pulmonary artery conduit. A systematic review and meta-analysis. Heart Lung Vessels 7, 304-310 (2015).
Yong, M. S. et al. Medium-term outcomes of bovine jugular vein graft and homograft conduits in children. ANZ J. Surg. 85, 381-385 (2015).
Hawkins, J. A. et al. Class I and class II anti-hla antibodies after implantation of cryopreserved allograft material in pediatric patients. J. Thorac. Cardiovasc. Surg. 119, 324-330 (2000).
Rüffer, A. et al. Mid-term experience with the Hancock porcine-valued Dacron conduit for right ventricular outflow tract reconstruction. Eur. J. Cardio-Thorac. Surg. Off. J. Eur. Assoc. Cardio-Thorac. Surg. 42, (2012).
Hill Garick D., Ginde Salil, Rios Rodrigo, Frommelt Peter C., & Hill Kevin D. Surgical Valvotomy Versus Balloon Valvuloplasty for Congenital Aortic Valve Stenosis: A Systematic Review and Meta-Analysis. J. Am. Heart Assoc. 5, e003931.
Cheatham John P. et al. Clinical and Hemodynamic Outcomes up to 7 Years After Transcatheter Pulmonary Valve Replacement in the US Melody Valve Investigational Device Exemption Trial. Circulation 131, 1960-1970 (2015).
Kenny Damien et al. 3-Year Outcomes of the Edwards SAPIEN Transcatheter Heart Valve for Conduit Failure in the Pulmonary Position From the COMPASSION Multicenter Clinical Trial. JACC Cardiovasc. Interv. 11, 1920-1929 (2018).
Bui, H., Harris, K., Li, E., Prawel, D. & James, S. The Development and Fabrication of Vapor Crosslinked Hyaluronan-Polyethylene Interpenetrating Polymer Network as a Biomaterial. ACS Appl. Mater. Interfaces 11, (2019).
Prawel, D. A. et al. Hemocompatibility and Hemodynamics of Novel Hyaluronan-Polyethylene Materials for Flexible Heart Valve Leaflets. Cardiovasc. Eng. Technol. 5, 70-81 (2014).
James, S. P. & Zhang, M. Hyaluronan (HA) esterification via acylation technique for moldable devices. (2010).
James, S. P., Zhang, M. & Beauregard, G. Outer layer having entanglement of hydrophobic polymer host and hydrophilic polymer guest. (2010).
James, S. P. et al. Glycosaminoglycan and synthetic polymer materials for blood-contacting applications. (2015) (e.g., US Published application No. 20180305528).
Brown, J. W., Ruzmetov, M., Okada, Y., Vijay, P. & Turrentine, M. W. Truncus arteriosus repair: outcomes, risk factors, reoperation and management. Eur. J. Cardiothorac. Surg. 20, 221-227 (2001).
Momenah Tarek S. et al. Extended Application of Percutaneous Pulmonary Valve Implantation. J. Am. Coll. Cardiol. 53, 1859-1863 (2009).
Hofferberth, Sophie C., et al. “A geometrically adaptable heart valve replacement.” Science translational medicine 12.531 (2020): eaay4006.
Li, Richard L., et al. “In Vitro Proof of Concept of a First-Generation Growth-Accommodating Heart Valved Conduit for Pediatric Use.” Macromolecular Bioscience (2023): 2300011.
U.S. Patent Publication No. 20160015516A1.
U.S. Pat. No. 5,5452,15
U.S. Patent Publication No. 20180271649A1.
Canadian Patent No. CA3077818
PCT Publication No. WO 2020/227359A1

1. A conduit device, comprising:
a thermoformed conduit comprising a first low melting point thermoplastic material suitable for thermoforming, the thermoformed conduit comprising a first end and a second end and defining a channel therebetween, wherein the conduit device is configured to be surgically implanted in a subject's heart or blood vessel.

2. The conduit device of claim 1, wherein the thermoformed conduit comprises a valve, and wherein the valve is positioned in the channel or at the second end.

3. The conduit device of claim 1, wherein the thermoformed conduit comprises:
one or more sewable rings, including a first sewable ring, the first sewable ring being coupled to a circumferential portion of the first end.

4. The conduit device of claim 1, wherein the thermoformed conduit comprises:
an inner layer that is made of the first low melting point thermoplastic material; and
an outer layer disposed over the first sewable ring, the second sewable ring, and the conduit such that the first and second sewable rings are fixed between the conduit and the outer layer, wherein the outer layer comprises a third low melting point thermoplastic material.

5. The conduit device of claim 1, wherein the thermoformed conduit comprises a hyaluronan additive.

6. The conduit device of claim 1, wherein at least one of the first low melting point thermoplastic material, the second thermoplastic material, and the third low melting point thermoplastic material comprises polyethylene.

7. The conduit device of claim 6, wherein the polyethylene comprises linear low-density polyethylene (LLDPE).

8. The conduit device of claim 1, wherein at least one of the first low melting point thermoplastic material, the second thermoplastic material, and the third low melting point thermoplastic material comprises polyethylene terephthalate (PET).

9. The conduit device of claim 1, wherein the thermoformed conduit comprises LLDPE and hyaluronic acid additive arranged in an interpenetrating network.

10. The conduit device of claim 1, wherein at least one of the first low melting point thermoplastic material, the second thermoplastic material, and the third low melting point thermoplastic material is a woven material.

11. The conduit device of claim 4, wherein at least one of the first low melting point thermoplastic material, the second thermoplastic material, and the third low melting point thermoplastic material is a non-woven material.

12. The conduit device of claim 4, wherein the thermoformed conduit is shaped to form a sinus.

13. The conduit device of claim 1, wherein the thermoformed conduit is calcification resistant or anti-thrombotic.

14. The conduit device of claim 1, wherein the thermoformed conduit is expandable to operate in a first installed configuration and a second modified configuration to have an expanded cross-sectional area compared to the first installed configuration.

15. The conduit device of claim 1, wherein the thermoformed conduit is configured to operate in a non-expandable state.

16. The conduit device of claim 1, wherein the thermoformed conduit includes any of the features of claims 24-34.

17. A method of fabricating a conduit device comprising: thermoforming a conduit device of claim 1 comprising a first low melting point thermoplastic material suitable for thermoforming, the thermoformed conduit comprising a first end and a second end and defining a channel therebetween, wherein the conduit device is configured to be surgically implanted in a subject's heart or blood vessel.

18. A method of fabricating a conduit device comprising: thermoforming a conduit device of claim 1 comprising an inner layer comprising a first low melting point thermoplastic material and an outer layer comprising a second low melting point thermoplastic material each suitable for thermoforming, wherein the thermoformed conduit comprises a first end and a second end and defines a channel therebetween, wherein the conduit device is configured to be surgically implanted in a subject's heart or blood vessel.

19.-22. (canceled)

23. An expandable conduit device comprising:
an expandable thermoformed conduit formed of a first low melting point thermoplastic material suitable of thermoforming, the thermoformed conduit comprising a first end and a second end and defining a channel therebetween,
wherein the expandable thermoformed conduit is configured to have (i) a first configuration having a first circumferential profile and (ii) a second configuration having a second circumferential profile, wherein the second circumferential profile has a larger cross-sectional area than the first circumferential profile,
wherein the expandable conduit device is configured to be surgically implanted in a subject's heart or blood vessel.

24.-34. (canceled)

35. A method comprising:
providing an expandable thermoformed conduit comprising a low melting point thermoplastic material suitable for thermoforming, the thermoformed conduit comprising a first end and a second end and defining a channel therebetween, wherein the conduit device is configured to be surgically embedded into a subject's heart, the expandable thermoformed conduit configured to have a first configuration having a first circumferential profile at a first position in the channel and a second configuration having a second circumferential profile at the first position, wherein the second circumferential profile has a larger cross-sectional area than the first circumferential profile;
implanting the expandable thermoformed conduit while the expandable thermoformed conduit is in the first configuration; and
mechanically inducing the expandable thermoformed conduit to the second configuration.

36.-41. (canceled)
